(19) United States
(12) Patent Application Publication
(10) Pub. No.: US 2025/0333383 A1
(43) Pub. Date: Oct. 30, 2025

(54) N-OXIDE COMPOUNDS AND USE THEREOF

(71) Applicant: SK BIOPHARMACEUTICALS CO., LTD., Gyeonggi-so (KR)

(72) Inventors: Young Soon Kang, Gyeonggi-do (KR); Ho Won Kang, Gyeonggi-do (KR); Yong Soo Kim, Gyeonggi-do (KR); Jin Sung Kim, Gyeonggi-do (KR); Min Hee Pang, Gyeonggi-do (KR); Ka Eun Lee, Gyeonggi-do (KR); Chi Yeon Rhee, Gyeonggi-do (KR); Han Ju Yi, Gyeonggi-do (KR); Sang Mi Chae, Gyeonggi-do (KR); Yong Do Kwak, Gyeonggi-do (KR); Hye Kyung Min, Gyeonggi-do (KR); Ji Won Lee, Gyeonggi-do (KR); Young Koo Jang, Gyeonggi-do (KR); Min Jae Jo, Gyeonggi-do (KR)

(73) Assignee: SK BIOPHARMACEUTICALS CO., LTD., Gyeonggi-so (KR)

(21) Appl. No.: 18/860,849

(22) PCT Filed: Apr. 27, 2023
(86) PCT No.: PCT/KR2023/005773

§ 371 Date: Oct. 28, 2024

Related U.S. Application Data
(60) Provisional application No. 63/335,779, filed on Apr. 28, 2022.

(51) Int. Cl. C07D 213/89; A61K 31/19; A61K 31/195; A61K 31/197; A61K 31/4425; A61K 33/00; A61K 45/06; A61P 25/00

(52) U.S. Cl. CPC C07D 213/89; A61K 31/19; A61K 31/195; A61K 31/197; A61K 31/4425; A61K 33/00; A61K 45/06; A61P 25/00

(57) ABSTRACT

The present disclosure provides a compound represented by Formula (I) or a pharmaceutically acceptable salt which are effective as a sodium channel blocker and a method of using the compound.

### TECHNICAL FIELD

The present disclosure generally relates to N-oxide compounds as sodium channel blockers, pharmaceutical compositions comprising the compounds, methods for treating pain including neuropathic pain in mammals by administering the sodium channel blockers to the mammals in need of treatment thereof. The present disclosure includes methods of using these inventive sodium channel blockers and processes for preparing the same.

### BACKGROUND ART

PAIN is defined by the International Association for the Study of Pain (IASP) as “an unpleasant sensory and emotional experience associated with actual or potential tissue damage, or described in terms of such damage”. In this 2^{nd} edition, two new terms have been introduced as Neuropathic Pain and Peripheral Neuropathic Pain. NEUROPATHIC PAIN is defined by the IASP as “pain initiated or caused by a primary lesion or dysfunction in the peripheral or central nervous system” (IASP, Classification of chronic pain (2^{nd} ed.), IASP Press, 2002, p 210).

The treatment of pain conditions is of a significant importance in medicine and there is currently a global need for new pain therapy. Especially, the urgent requirement for a specific treatment of pain conditions or as well a treatment of specific pain conditions is reported in a lot of scientific works. Even though pain is always subjective, its causes or syndromes can be classified into several subtypes.

Neuropathic pain which in the past years has developed into a major health problem in broad areas of the population also needs a very specific treatment, especially considering that any treatment of neuropathic pain is extremely sensitive to the causes behind the pain. So, in a majority of cases a substance being able to treat one subtype of neuropathic pain is not—or is at least not necessarily—able to treat other specific subtypes due to the highly diverse nature of this generalized symptom called neuropathic pain.

So far, there are more than 100 known types of neuropathic pain. Common acquired causes include viral infections, diabetes, injury, autoimmune disease, and nutritional deficiency. Some neuropathic pain can also be inherited or caused by genetic mutations. For the purpose of this invention, the subtypes can be included under this heading or to be treated as synonymous.

Voltage-gated sodium channels (VGSCs) control the flow of sodium ions that can trigger excitability of pain-sensing nociceptors in the peripheral nervous system. In humans, nine VGSCs have now been identified, and some have been linked to genes that alter their function. Sodium channels play an important role in painful neuropathy.

Especially, gene mutations have now been linked to sodium channels Nav1.7, Nay1.8, and Nav1.9. Gain of function mutation in the gene SCN9A has been linked to Nay1.7 and a painful neuropathic disorder called inherited erythromelalgia (IEM). The gene SCN10A has been linked to Nay1.8 and small fiber neuropathy, a condition that causes severe pain attacks in the hands or feet. It may also cause autonomic pain symptoms such as palpitations, bowel problems, and abnormal sweating. Nay1.9 has also been linked to mutation that causes painful neuropathy.

Thiazole compounds of the present disclosure provide a new form of treatment for neuropathic pain as sodium channel blockers.

For an example, U.S. Pat. No. 8,822,463 describes that methylcyclohexane compounds represented by the following formula are effective in treating neuropathic pain.

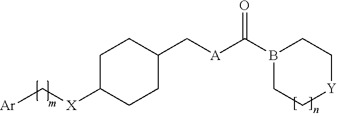

For another example, U.S. Pat. No. 8,541,409 mentions carbamoyloxy arylalkanoyl arylpiperazine compounds useful as analgesic agents as follows.

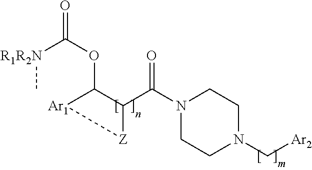

### DISCLOSURE OF INVENTION

### Technical Problem

The purpose of the present disclosure is the provision of a compound useful as a sodium channel blocker.

### Solution to Problem

The present disclosure provides a novel compound of the following Formula (I), or an optical isomer, a stereoisomer or an isotopic variant thereof, or a pharmaceutically acceptable salt thereof:

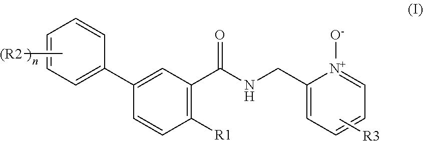

wherein
R1 is hydrogen, halogen, alkyl, alkoxy or cycloalkyl;
R2 is hydrogen, halogen, alkyl, alkoxy, cycloalkyl or 4- to 10-membered heterocycloalkyl which includes one or more heteroatoms selected from N, O and S;
R3 is hydrogen, halogen, alkyl, alkoxy or cycloalkyl; and
n is an integer of 0 to 4,
wherein the alkyl, alkoxy and cycloalkyl are optionally substituted with one or more substituents selected from halogen and hydroxy.

The compounds of Formula (I) are useful in inhibiting sodium channel inactivated state.

In some embodiments, the compound of Formula (I) is a compound of the following Formula (II):

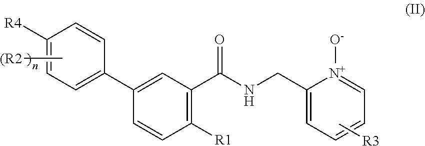

wherein R1, R2, R3 and n are as defined above; and
R4 is hydrogen, halogen, alkyl, alkoxy, cycloalkyl or 4- to 10-membered heterocycloalkyl which includes one or more heteroatoms selected from N, O and S.

In another embodiment, there is provided a pharmaceutical composition comprising a therapeutically effective amount of one or more compounds described herein and a pharmaceutically acceptable carrier.

In yet another embodiment, there is provided a method of treating or preventing pain including neuropathic pain in a mammal in need thereof by administering a therapeutically effective amount of the compound represented by Formula (I) or (II), or an optical isomer, a stereoisomer or an isotopic variant thereof, or a pharmaceutically acceptable salt thereof.

### Advantageous Effects of Invention

The compounds of the present disclosure as sodium channel blockers are useful to treat pain including neuropathic pain.

### MODE FOR THE INVENTION

The following description is merely exemplary in nature and is not intended to limit the present disclosure, application, or uses.

### Definitions

“Alkoxy” is RO— where R is alkyl. Non-limiting examples of alkoxy groups include methoxy, ethoxy and propoxy. In an embodiment, the alkoxy group is methoxy.

“Alkyl” refers to a straight or branched chain saturated hydrocarbyl group. In an embodiment, alkyl has from 1 to 12 carbon atoms. In some embodiments, alkyl is a C₁-C₁₀ alkyl group, a C₁-C₆ alkyl group or a C₁-C₄ alkyl group. Examples of alkyl groups include, but are not limited to, methyl, ethyl, propyl, isopropyl, butyl, isobutyl, t-butyl, pentyl, hexyl, heptyl, octyl, nonyl and decyl.

“Cycloalkyl” refers to a saturated or partially saturated, monocyclic, fused polycyclic, or spiro polycyclic carbocycle having from 3 to 12 ring atoms per carbocycle.

“Halo” or “Halogen” refers to fluoro (—F), chloro (—Cl), bromo (—Br), or iodo (—I).

When a particular group is “substituted” (e.g., alkoxy, alkyl, cycloalkyl, etc.), that group may have one or more substituents, for example, from one to five substituents, or from one to three substituents, or one to two substituents, independently selected from the list of substituents.

“Pharmaceutically acceptable” means suitable for use in pharmaceutical preparations, generally considered as safe for such use, officially approved by a regulatory agency of a national or state government for such use, or being listed in the U.S. Pharmacopoeia or other generally recognized pharmacopoeia for use in animals, and more particularly in humans.

“Pharmaceutically acceptable carrier” refers to a diluent, adjuvant, excipient, or carrier that alone or together provides a carrier or vehicle with which a compound or compounds of the disclosure is formulated and/or administered, and in which every ingredient or the carrier as a whole is pharmaceutically acceptable.

“Pharmaceutically acceptable salt” refers to a salt which may enhance desired pharmacological activity. Examples of pharmaceutically-acceptable salts include acid addition salts formed with inorganic or organic acids, metal salts and amine salts. Examples of acid addition salts formed with inorganic acids include salts with hydrochloric acid, hydrobromic acid, sulfuric acid, nitric acid and phosphoric acid. Examples of acid addition salts formed with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxy-benzoyl)-benzoic acid, cinnamic acid, mandelic acid, methanesulfonic acid, ethanesulfonic acid, 1,2-ethanedisulfonic acid, 2-hydroxyethane-sulfonic acid, benzenesulfonic acid, p-chlorobenzenesulfonic acid, 2-naphthalenesulfonic acid, p-toluenesulfonic acid, camphorsulfonic acid, 4-methyl-bicyclo[2.2.2]oct-2-ene-1-carboxylic acid, gluco-heptonic acid, 4,4′-methylenebis(3-hydroxy-2-naphthoic) acid, 3-phenylpropionic acid, trimethyl-acetic acid, tertiary butylacetic acid, lauryl sulfuric acid, gluconic acid, glutamic acid, hydroxy-naphthoic acids, salicylic acid, stearic acid and muconic acid. Examples of metal salts include salts with sodium, potassium, calcium, magnesium, aluminum, iron, and zinc ions. Examples of amine salts include salts with ammonia and organic nitrogenous bases strong enough to form salts with carboxylic acids.

“Therapeutically effective amount” refers to an amount of a compound that, when administered to a subject for treating a disease, is sufficient to effect treatment for the disease. “Therapeutically effective amount” can vary depending on the compound, the disease and its severity, the age, the weight, etc. of the subject to be treated.

Embraced herein, where applicable, are permissible isomers such as tautomers, racemates, enantiomers, diastereomers, atropisomers, and isotopic variants.

### Compounds

This disclosure provides a compound of the following Formula (I), or an optical isomer, a stereoisomer or an isotopic variant thereof, or a pharmaceutically acceptable salt thereof:

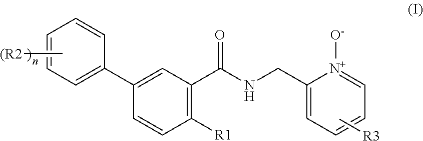

wherein
R1 is hydrogen, halogen, alkyl, alkoxy or cycloalkyl;
R2 is hydrogen, halogen, alkyl, alkoxy, cycloalkyl or 4- to 10-membered heterocycloalkyl which includes one or more heteroatoms selected from N, O and S;
R3 is hydrogen, halogen, alkyl, alkoxy or cycloalkyl; and
n is an integer of 0 to 4,
wherein the alkyl, alkoxy and cycloalkyl are optionally substituted with one or more substituents selected from halogen and hydroxy.

In an embodiment, R1 is hydrogen, halogen, C₁-C₆ alkyl, C₁-C₆ alkoxy or C₃-C₁₀ cycloalkyl; R2 is hydrogen, halogen, C₁-C₆ alkyl, C₁-C₆ alkoxy, C₃-C₁₀ cycloalkyl or 4- to 10-membered heterocycloalkyl which includes 1 to 4 heteroatoms selected from N, O and S; R3 is hydrogen, halogen, C₁-C₆ alkyl, C₁-C₆ alkoxy or C₃-C₁₀ cycloalkyl; and n is an integer of 0 to 4, wherein the alkyl, alkoxy and cycloalkyl are optionally substituted with 1 to 4 substituents selected from halogen and hydroxy.

In another embodiment, R1 is hydrogen, halogen, C₁-C₄ alkyl, C₁-C₄ alkoxy or C₃-C₆ cycloalkyl; R2 is hydrogen, halogen, C₁-C₄ alkyl, C₁-C₄ alkoxy, C₃-C₆ cycloalkyl or 4- to 10-membered heterocycloalkyl which includes 1 to 4 heteroatoms selected from N, O and S; R3 is hydrogen, halogen, C₁-C₄ alkyl, C₁-C₄ alkoxy or C₃-C₆ cycloalkyl; and n is an integer of 0 to 4, wherein the alkyl, alkoxy and cycloalkyl are optionally substituted with 1 to 4 substituents selected from halogen and hydroxy.

In another embodiment, R1 is hydrogen or halogen.

In another embodiment, R2 is hydrogen, halogen, C₁-C₆ alkyl, C₁-C₆ alkoxy or C₃-C₁₀ cycloalkyl; wherein the alkyl, alkoxy and cycloalkyl are optionally substituted with 1 to 4 substituents selected from halogen and hydroxy.

In another embodiment, R3 is hydrogen or halogen.

In another embodiment, there is provided a compound of the following Formula (II), or optical isomer, stereoisomer or isotopic variant thereof, or a pharmaceutically acceptable salt thereof:

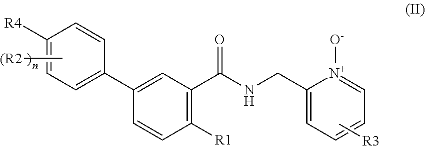

wherein
R1 is hydrogen, halogen, alkyl, alkoxy or cycloalkyl;
R2 is hydrogen, halogen, alkyl, alkoxy, cycloalkyl or 4- to 10-membered heterocycloalkyl which includes one or more heteroatoms selected from N, O and S;
R3 is hydrogen, halogen, alkyl, alkoxy or cycloalkyl;
R4 is hydrogen, halogen, alkyl, alkoxy, cycloalkyl or 4- to 10-membered heterocycloalkyl which includes one or more heteroatoms selected from N, O and S; and
n is an integer of 0 to 3,
wherein the alkyl, alkoxy and cycloalkyl are optionally substituted with one or more substituents selected from halogen and hydroxy.

In another embodiment, R1 is hydrogen, halogen, C₁-C₆ alkyl, C₁-C₆ alkoxy or C₃-C₁₀ cycloalkyl; R2 is hydrogen, halogen, C₁-C₆ alkyl, C₁-C₆ alkoxy, C₃-C₁₀ cycloalkyl or 4- to 10-membered heterocycloalkyl which includes 1 to 4 heteroatoms selected from N, O and S; R3 is hydrogen, halogen, C₁-C₆ alkyl, C₁-C₆ alkoxy or C₃-C₁₀ cycloalkyl; R4 is hydrogen, halogen, C₁-C₆ alkyl, C₁-C₆ alkoxy, C₃-C₁₀ cycloalkyl or 4- to 10-membered heterocycloalkyl which includes 1 to 4 heteroatoms selected from N, O and S; and n is an integer of 0 to 3, wherein the alkyl, alkoxy and cycloalkyl are optionally substituted with 1 to 4 substituents selected from halogen and hydroxy.

In another embodiment, R1 is hydrogen, halogen, C₁-C₄ alkyl, C₁-C₄ alkoxy or C₃-C₆ cycloalkyl; R2 is hydrogen, halogen, C₁-C₄ alkyl, C₁-C₄ alkoxy, C₃-C₆ cycloalkyl or 4- to 10-membered heterocycloalkyl which includes 1 to 4 heteroatoms selected from N, O and S; R3 is hydrogen, halogen, C₁-C₄ alkyl, C₁-C₄ alkoxy or C₃-C₆ cycloalkyl; R4 is hydrogen, halogen, C₁-C₄ alkyl, C₁-C₄ alkoxy, C₃-C₆ cycloalkyl or 4- to 10-membered heterocycloalkyl which includes 1 to 4 heteroatoms selected from N, O and S; and n is an integer of 0 to 3, wherein the alkyl, alkoxy and cycloalkyl are optionally substituted with 1 to 4 substituents selected from halogen and hydroxy.

In another embodiment, R1 is hydrogen or halogen.

In another embodiment, R2 is hydrogen, halogen, C₁-C₆ alkyl or C₁-C₆ alkoxy; wherein the alkyl and alkoxy are optionally substituted with 1 to 4 substituents selected from halogen and hydroxy.

In another embodiment, R3 is hydrogen or halogen.

In another embodiment, R4 is hydrogen, halogen, C₁-C₆ alkyl, C₁-C₆ alkoxy or C₃-C₁₀ cycloalkyl; wherein the alkyl, alkoxy and cycloalkyl are optionally substituted with 1 to 4 substituents selected from halogen and hydroxy.

Examples of Formula (I) includes, but are not limited to, the following compounds:
N-[(1-oxidopyridin-1-ium-2-yl)methyl]-3-[4-(trifluoromethyl)phenyl]benzamide;
3-(4-chlorophenyl)-N-[(1-oxidopyridin-1-ium-2-yl)methyl]benzamide;
N-[(1-oxidopyridin-1-ium-2-yl)methyl]-3-[4-(trifluoromethoxy)phenyl]benzamide;
3-(3-chlorophenyl)-N-[(1-oxidopyridin-1-ium-2-yl)methyl]benzamide;
N-[(1-oxidopyridin-1-ium-2-yl)methyl]-3-[3-(trifluoromethyl)phenyl]benzamide;
3-(3,4-dichlorophenyl)-N-[(1-oxidopyridin-1-ium-2-yl)methyl]benzamide;
3-(3,5-dichlorophenyl)-N-[(1-oxidopyridin-1-ium-2-yl)methyl]benzamide;
N-[(1-oxidopyridin-1-ium-2-yl)methyl]-3-[3-(trifluoromethoxy)phenyl]benzamide;
3-(2,4-dichlorophenyl)-N-[(1-oxidopyridin-1-ium-2-yl)methyl]benzamide;
3-(2,5-dichlorophenyl)-N-[(1-oxidopyridin-1-ium-2-yl)methyl]benzamide;
3-[2,4-bis(trifluoromethyl)phenyl]-N-[(1-oxidopyridin-1-ium-2-yl)methyl]benzamide
N-[(1-oxidopyridin-1-ium-2-yl)methyl]-3-phenyl-benzamide;
3-(4-tert-butylphenyl)-N-[(1-oxidopyridin-1-ium-2-yl)methyl]benzamide;
N-[(1-oxidopyridin-1-ium-2-yl)methyl]-3-(p-tolyl)benzamide;
3-(4-chloro-2-methyl-phenyl)-N-[(1-oxidopyridin-1-ium-2-yl)methyl]benzamide;
3-[2-fluoro-4-(trifluoromethyl)phenyl]-N-[(1-oxidopyridin-1-ium-2-yl)methyl]benzamide;
3-(3-chloro-4-fluoro-phenyl)-N-[(1-oxidopyridin-1-ium-2-yl)methyl]benzamide;
3-(4-fluorophenyl)-N-[(1-oxidopyridin-1-ium-2-yl)methyl]benzamide;
3-(3,4-difluorophenyl)-N-[(1-oxidopyridin-1-ium-2-yl)methyl]benzamide;
3-(2-chlorophenyl)-N-[(1-oxidopyridin-1-ium-2-yl)methyl]benzamide;
N-[(1-oxidopyridin-1-ium-2-yl)methyl]-3-[2-(trifluoromethyl)phenyl]benzamide;
3-(4-fluoro-3-methyl-phenyl)-N-[(1-oxidopyridin-1-ium-2-yl)methyl]benzamide;
3-(m-tolyl)-N-[(1-oxidopyridin-1-ium-2-yl)methyl]benzamide;
3-(4-isopropylphenyl)-N-[(1-oxidopyridin-1-ium-2-yl)methyl]benzamide;
3-(2-fluorophenyl)-N-[(1-oxidopyridin-1-ium-2-yl)methyl]benzamide;
3-(4-chloro-2-methoxy-phenyl)-N-[(1-oxidopyridin-1-ium-2-yl)methyl]benzamide;
3-[4-chloro-2-(trifluoromethyl)phenyl]-N-[(1-oxidopyridin-1-ium-2-yl)methyl]benzamide;
3-(4-chloro-3-methyl-phenyl)-N-[(1-oxidopyridin-1-ium-2-yl)methyl]benzamide;
3-(3-chloro-5-fluoro-phenyl)-N-[(1-oxidopyridin-1-ium-2-yl)methyl]benzamide;
3-(4-chloro-3-fluoro-phenyl)-N-[(1-oxidopyridin-1-ium-2-yl)methyl]benzamide;
3-(2,4-difluorophenyl)-N-[(1-oxidopyridin-1-ium-2-yl)methyl]benzamide;
3-(3,5-difluorophenyl)-N-[(1-oxidopyridin-1-ium-2-yl)methyl]benzamide;
N-[(1-oxidopyridin-1-ium-2-yl)methyl]-3-(3,4,5-trifluorophenyl)benzamide;
3-(2,3-difluorophenyl)-N-[(1-oxidopyridin-1-ium-2-yl)methyl]benzamide;
3-(3-isopropylphenyl)-N-[(1-oxidopyridin-1-ium-2-yl)methyl]benzamide;
3-(4-cyclopropylphenyl)-N-[(1-oxidopyridin-1-ium-2-yl)methyl]benzamide;
3-[4-(difluoromethoxy)phenyl]-N-[(1-oxidopyridin-1-ium-2-yl)methyl]benzamide;
3-[2-methyl-4-(trifluoromethyl)phenyl]-N-[(1-oxidopyridin-1-ium-2-yl)methyl]benzamide;
3-[4-fluoro-3-(trifluoromethyl)phenyl]-N-[(1-oxidopyridin-1-ium-2-yl)methyl]benzamide;
3-(2-methoxyphenyl)-N-[(1-oxidopyridin-1-ium-2-yl)methyl]benzamide;
3-[3-fluoro-5-(trifluoromethyl)phenyl]-N-[(1-oxidopyridin-1-ium-2-yl)methyl]benzamide;
3-[3-fluoro-4-(trifluoromethyl)phenyl]-N-[(1-oxidopyridin-1-ium-2-yl)methyl]benzamide;
3-[3-methyl-4-(trifluoromethyl)phenyl]-N-[(1-oxidopyridin-1-ium-2-yl)methyl]benzamide;
3-[4-(1-hydroxy-1-methyl-ethyl)phenyl]-N-[(1-oxidopyridin-1-ium-2-yl)methyl]benz amide;
3-[3-methoxy-5-(trifluoromethyl)phenyl]-N-[(1-oxidopyridin-1-ium-2-yl)methyl]benzamide;
3-[2-methoxy-4-(trifluoromethoxy)phenyl]-N-[(1-oxidopyridin-1-ium-2-yl)methyl]benzamide;
3-(4,5-difluoro-2-methoxy-phenyl)-N-[(1-oxidopyridin-1-ium-2-yl)methyl]benzamide;
3-(4-chloro-3-methoxy-phenyl)-N-[(1-oxidopyridin-1-ium-2-yl)methyl]benzamide;
3-[3-methoxy-4-(trifluoromethyl)phenyl]-N-[(1-oxidopyridin-1-ium-2-yl)methyl]benzamide;
3-[4-methoxy-2-(trifluoromethyl)phenyl]-N-[(1-oxidopyridin-1-ium-2-yl)methyl]benzamide;
5-(4-chlorophenyl)-2-fluoro-N-[(1-oxidopyridin-1-ium-2-yl)methyl]benzamide;
5-(3-chlorophenyl)-2-fluoro-N-[(1-oxidopyridin-1-ium-2-yl)methyl]benzamide;
5-(4-chloro-2-methoxy-phenyl)-2-fluoro-N-[(1-oxidopyridin-1-ium-2-yl)methyl]benz amide;
5-(3,4-difluorophenyl)-2-fluoro-N-[(1-oxidopyridin-1-ium-2-yl)methyl]benzamide;
5-[4-chloro-2-(trifluoromethyl)phenyl]-2-fluoro-N-[(1-oxidopyridin-1-ium-2-yl)methyl]benzamide;
2-fluoro-5-[2-methoxy-4-(trifluoromethoxy)phenyl]-N-[(1-oxidopyridin-1-ium-2-yl)methyl]benzamide;
2-fluoro-N-[(1-oxidopyridin-1-ium-2-yl)methyl]-5-[4-(trifluoromethyl)phenyl]benzamide;
2-fluoro-N-[(1-oxidopyridin-1-ium-2-yl)methyl]-5-[4-(trifluoromethoxy)phenyl]benz amide;
2-fluoro-5-[3-methoxy-4-(trifluoromethyl)phenyl]-N-[(1-oxidopyridin-1-ium-2-yl)methyl]benzamide;
2-fluoro-5-[3-fluoro-4-(trifluoromethyl)phenyl]-N-[(1-oxidopyridin-1-ium-2-yl)methyl]benzamide;
3-(4-chlorophenyl)-N-[(5-fluoro-1-oxido-pyridin-1-ium-2-yl)methyl]benzamide; and
2-chloro-5-(4-chlorophenyl)-N-[(1-oxidopyridin-1-ium-2-yl)methyl]benzamide.

### Synthesis of Compounds

In some embodiments, the compounds of Formula (I) can be prepared by the synthetic method of Scheme I, II or, III as described below.

When X and Y are H, the desired compound may be prepared by the synthetic method described in Scheme I:

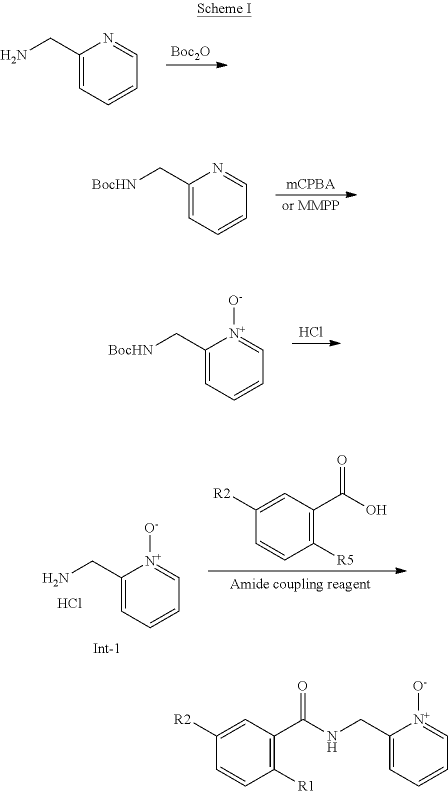

As shown in Scheme I, (1-oxidopyridin-1-ium-2-yl)methanamine hydrochloride (Int-1) was synthesize in by general protection of benzylamine, oxidation of pyridine and deprotection of Boc group. The desired target compounds were obtained by the general amide coupling of Int-1 with the corresponding carboxylic acid which is commercially available.

Details of the reaction conditions described in Scheme I are as follows. To a solution of 2-pyridylmethanamine in DCM was treated Boc₂O at zero degree. The concentration of 2-pyridylmethanamine as a starting material was about 0.001 to 100 mole. This reaction was preferably carried out at an ambient temperature. Oxidation of Boc protected compound was performed by oxidant such as mCPBA or MMPP. Oxidant was used about 3 equivalents. This reaction was preferably carried out in MeOH at 60° C. for 3 hours. Target compounds were synthesized from Int-1 by deprotection of Boc group using excess of HCl solution in organic solvent followed by amide coupling as represented in Scheme I.

When R3 is not H, the desired compound may be prepared by the synthetic method described in Scheme II:

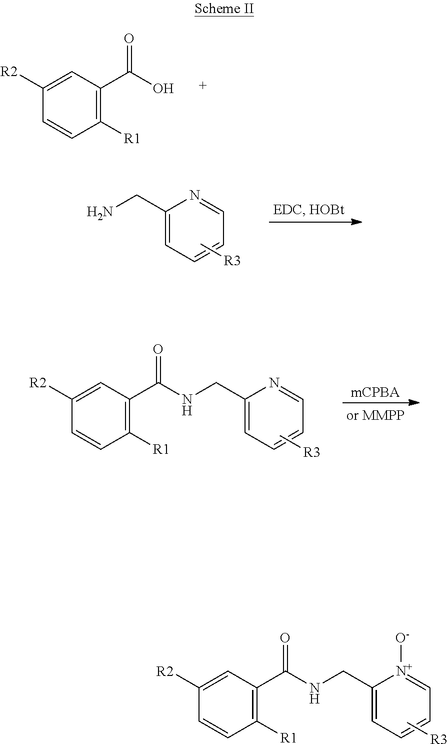

As shown in Scheme II, Substituent 2-pyridylmethanamine and corresponding carboxylic acid were coupled with amide coupling reagents such as EDC and HOBt. The target compounds were synthesized in oxidation of pyridine.

Details of the reaction conditions described in Scheme II are the same as depicted scheme I. The concentration of each carboxylic acid as starting material was between about 0.01 to 0.1 mole.

When the corresponding carboxylic acids were not commercially available, the desired compound may be prepared by the synthetic method described in Scheme III:

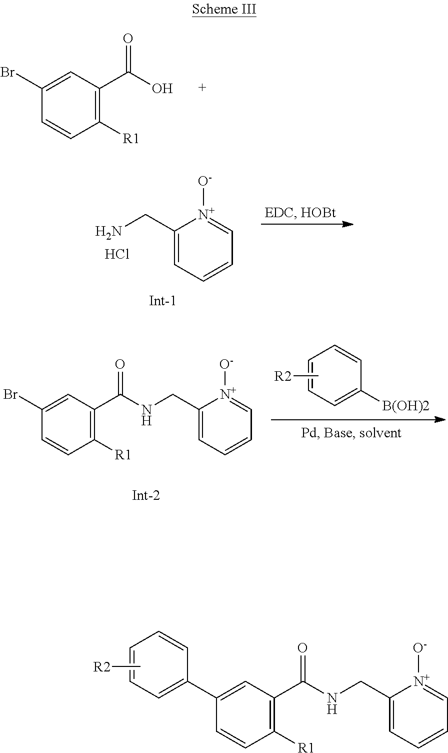

As shown in Scheme III, bromo-carboxylic acid compounds and Int-1 were reacted with amide coupling reagents to give Int-2. Palladium catalyst reaction with proper base and solvent could get the final target compounds.

Details of the reaction conditions described in Scheme III are as follows. Amide coupling explained above. Palladium catalyst metal reaction was performed. General reaction condition was Pd(dppf)Cl₂ as a metal, Na₂CO₃ as a base, and DME as a solvent. This metal reaction was carried out by various reaction conditions due to reactivity of two starting materials.

### Pharmaceutical Compositions

In one embodiment, there is provided a pharmaceutical composition comprising, in addition to one or more compounds described herein, a pharmaceutically acceptable carrier. In various embodiments, the carrier comprises a diluent, adjuvant, excipient, other additive, or a combination of additive that separately or together provide a carrier in which the compositions can be formulated or administered. The composition can take any suitable form for the desired route of administration. Where the composition is to be administered orally, any suitable orally deliverable dosage form can be used, including, without limitation, tablets, capsules (solid- or liquid-filled), powders, granules, syrups and other liquids, elixirs, inhalants, troches, lozenges, and solutions. Injectable compositions or iv infusions are also provided in the form of solutions, suspensions, and emulsions.

In particular embodiments, the pharmaceutical composition is an oral formulation. Since the compounds of Formula (I) absorb well orally, it is generally unnecessary to resort to parenteral administration. For oral administration, the compound is preferably formulated with a pharmaceutically acceptable carrier. The ratio of the carrier to the compound would not be critical to the pharmacological effects of the formulation, and the ratio can vary considerably depending on formulating conditions. In tableting, various edible pharmaceutical carriers or the mixture thereof can be included therein. A suitable carrier, for example, is a mixture of lactose, dibasic calcium phosphate and/or corn starch. Other pharmaceutically acceptable ingredients can be further added, including lubricants such as magnesium stearate.

A pharmaceutical composition according to the present disclosure may contain one or more additional therapeutic agents, for example, to increase the efficacy or decrease the side effects. In an embodiment, the pharmaceutical composition includes one or more active ingredients effective to treat or prevent neuropathic pain, by including anticonvulants such as gabapentin, pregabalin, carbamazepine, antidepressants such as duloxetine, milnacipran, and analgesics such as opioids, NSAIDs, lidocaine patches and capsaicin patches.

### Medical Utilities

Yet another aspect of the present disclosure is to provide method of treating or preventing a disease or condition mediated by sodium channels, comprising administering to said mammals a therapeutically effective amount of one or more compounds of Formula (I) or Formula (II), or optical isomer, stereoisomer or isotopic variant thereof, or a pharmaceutically acceptable salt thereof. In another embodiment, the sodium channel may be one or more selected from the group of consisting of NaV1.1, NaV1.2, NaV1.3, NaV 1.4, NaV1.5, NaV 1.6, NaV1.7, NaV 1.8, and NaV1.9.

In another embodiment, the disease or condition may be pain. In another embodiment, the pain may be selected from the group consisting of pain, acute pain, chronic pain, neuropathic pain, inflammatory pain, visceral pain, nociceptive pain including post-surgical pain, and mixed pain types involving the viscera, gastrointestinal tract, cranial structures, musculoskeletal system, spine, urogenital system, cardiovascular system and CNS (central nervous system), including cancer pain, back pain, orofacial pain and chemo-induced pain.

In view of the above-described mechanisms of action, the compounds of the present invention are useful in the prevention or treatment of neuropathic pain. Neuropathic pain syndromes include, and are not limited to: small fiber neuropathy, lumbosacral radiculopathy, peripheral neuropathic pain, diabetic neuropathy; sciatica; non-specific lower back pain; multiple sclerosis pain; fibromyalgia; HIV-related neuropathy; neuralgia, such as post-herpetic neuralgia and trigeminal neuralgia, Morton's neuralgia, causalgia; and pain resulting from physical trauma, amputation, phantom limb, cancer, toxins or chronic inflammatory conditions; central pain such as the one observed in thalamic syndromes, mixed central and peripheral forms of pain such as complex regional pain syndromes (CRPS) also called reflex sympathetic dystrophies.

The compounds of the invention are also useful for the treatment or prevention of chronic pain. Chronic pain includes, and is not limited to, chronic pain caused by inflammation or an inflammatory-related condition, osteoarthritis, rheumatoid arthritis, acute injury or trauma, upper back pain or lower back pain (resulting from systematic, regional or primary spine disease such as radiculopathy), bone pain (due to osteoarthritis, osteoporosis, bone metastasis or unknown reasons), pelvic pain, spinal cord injury-associated pain, cardiac chest pain, non-cardiac chest pain, central poststroke pain, myofascial pain, sickle cell pain, cancer pain, Fabry's disease, AIDS pain, geriatric pain or pain caused by headache, temporomandibular joint syndrome, gout, fibrosis or thoracic outlet syndromes, in particular rheumatoid arthritis and osteoarthritis.

The compounds of the invention are also useful in the treatment or prevention of acute pain caused by acute injury, illness, sports-medicine injuries, carpal tunnel syndrome, burns, musculoskeletal sprains and strains, musculotendinous strain, cervicobrachial pain syndromes, dyspepsis, gastric ulcer, duodenal ulcer, dysmenorrhoea, endometriosis or surgery (such as open heart or bypass surgery), post-operative pain, kidney stone pain, gallbladder pain, gallstone pain, obstetric pain or dental pain.

The compounds of the invention are also useful in the treatment or prevention of headaches, migraine, tension type headache, transformed migraine or evolutive headache, cluster headache, as well as secondary headache disorders, such as the ones derived from infections, metabolic disorders or other systemic illnesses and other acute headaches, paroxysmal hemicrania and the like, resulting from a worsening of the above mentioned primary and secondary headaches.

In another embodiment, the disease or condition may be selected from the group consisting of neurological disorders, neurodegenerative disorders, inflammatory disorders, gastrointestinal (GI) tract disorders, disorders of the genito-urinary tract, psychiatric disorders, cardiovascular disorders and neuromuscular disorders. The compounds of the invention are also useful for the treatment or prevention of neurological disorders such as epilepsy including simple partial seizure, complex partial seizure, secondary generalized seizure, further including absence seizure, myoclonic seizure, clonic seizure, tonic seizure, tonic clonic seizure and atonic seizure.

The compounds of the invention are also useful for the treatment or prevention of neurodegenerative disorders of various origins including Alzheimer Disease, Parkinson Disease and other dementia conditions such as Lewys body, fronto-temporal dementia and taupathies; multiple sclerosis, amyotrophic lateral sclerosis and other parkinsonian syndromes; other spino cerebellar degeneration and Charcot-Marie-Tooth neuropathy, traumatic brain injury, stroke and cerebral ischemia.

The compounds of the invention inhibit inflammatory processes affecting all body systems. Therefore are useful in the treatment or prevention of inflammatory processes of the muscular-skeletal system of which the following is a list of examples but it is not comprehensive of all target disorders: arthritic conditions such as alkylosing spondylitis, cervical arthritis, fibromyalgia, gut, juvenile rheumatoid arthritis, lumbosacral arthritis, osteoarthritis, osteoporosis, psoriatic arthritis, rheumatic disease; disorders affecting skin and related tissues: eczema, psoriasis, pruritus, dermatitis and inflammatory conditions such as sunburn; disorders of the respiratory system: asthma, allergic rhinitis and respiratory distress syndrome, lung disorders in which inflammation is involved such as asthma and bronchitis; chronic obstructive pulmonary disease; disorders of the immune and endocrinological systems: periarthritis nodosa, thyroiditis, aplastic anaemia, sclerodoma, myasthenia gravis, multiple sclerosis and other demyelinizating disorders, encephalomyelitis, sarcoidosis, nephritic syndrome, Bechet's syndrome, polymyositis, gingivitis.

The compounds of the invention are also useful in the treatment or prevention of gastrointestinal (GI) tract disorders such as inflammatory bowel disorders including but not limited to ulcerative colitis, Crohn's disease, ileitis, pancreatitis, proctitis, celiac disease, enteropathies, microscopic or collagenous colitis, eosinophilic gastroenteritis, or pouchitis resulting after proctocolectomy and post ileonatal anastomosis, and irritable bowel syndrome including any disorders associated with abdominal pain and/or abdominal discomfort such as pylorospasm, nervous indigestion, spastic colon, spastic colitis, spastic bowel, intestinal neurosis, functional colitis, mucous colitis, laxative colitis and functional dyspepsia; but also for treatment of atrophic gastritis, gastritis varialoforme, ulcerative colitis, peptic ulceration, pyresis, and other damage to the GI tract, for example, by Helicobacter pylori, gastroesophageal reflux disease, gastroparesis, such as diabetic gastroparesis; and other functional bowel disorders, such as non-ulcerative dyspepsia (NUD); emesis, diarrhoea, and visceral inflammation.

The compounds of the invention are also useful in the treatment or prevention of disorders of the genito-urinary tract such as bladder dysfunction, overactive bladder, prostatitis (chronic bacterial and chronic non-bacterial prostatitis), prostadynia, interstitial cystitis, urinary incontinence and benign prostatic hyperplasia, annexities, pelvic inflammation, bartholinities and vaginitis. In particular, overactive bladder and urinary incontinence.

The compounds of the invention are also useful for the treatment or prevention of psychiatric disorders such as bipolar depression, mood disorder, bipolar disorder, anxiety and depression.

The compounds of the invention are also useful for the treatment or prevention of cardiovascular or neuromuscular disorders such as tachyarrhythmias, myotonia, arrhythmia, movement disorders, neuroendocrine disorders and ataxia.

### EXAMPLES

### Example 1: N-[(1-oxidopyridin-1-ium-2-yl)methyl]-3-[4-(trifluoromethyl)phenyl]benzamide

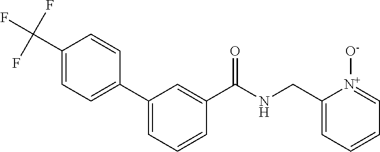 [SMILES: O=C(NCc1cccc[n+]1[O-])c1cccc(-c2ccc(C(F)(F)F)cc2)c1]

To a solution of 3-bromo-N-[(1-oxidopyridin-1-ium-2-yl)methyl]benzamide (0.95 mmol) in DME, 4-trifluoromethylphenyl boronic acid (1.91 mmol), pd(dppf)Cl₂ (0.24 mmol), and 2N Na₂CO₃ were added. The reaction mixture was stirred at 100° C. for 15 min. The reaction mixture was cooled to room temperature and filtered to remove palladium residues. The resulting mixture was extracted with DCM, washed with water and brine. The organic layer was dried over magnesium sulfate and concentrated in vacuo. The crude was purified by flash column chromatography to provide N-[(1-oxidopyridin-1-ium-2-yl)methyl]-3-[4-(trifluoromethyl)phenyl]benzamide.

1H-NMR (CDCl₃, 500 MHz) δ=8.28-8.25 (m, 1H), 8.17 (s, 1H), 7.98-7.96 (m, 1H), 7.82-7.80 (m, 1H), 7.73-7.71 (m, 4H), 7.58 (dd, J=2.0, 7.5 Hz, 1H), 7.54-7.51 (m, 1H), 7.33-7.28 (m, 2H), 4.87 (d, J=6.0 Hz, 2H).

### Example 2: 3-(4-chlorophenyl)-N-[(1-oxidopyridin-1-ium-2-yl)methyl]benzamide

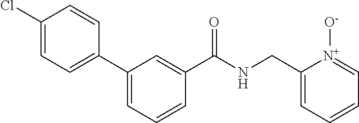 [SMILES: O=C(NCc1cccc[n+]1[O-])c1cccc(-c2ccc(Cl)cc2)c1]

The procedure given in Example 1 was followed using 4-chlorophenyl boronic acid as a reactant, instead of 4-trifluoromethylphenyl boronic acid, to give 3-(4-chlorophenyl)-N-[(1-oxidopyridin-1-ium-2-yl)methyl]benzamide.

1H-NMR (CDCl₃, 500 MHz) δ=8.25 (d, J=6.0 Hz, 1H), 8.00 (s, 1H), 7.92 (brs, 1H, NH), 7.75 (d, J=8.0 Hz, 1H), 7.66 (d, J=7.5 Hz, 1H), 7.56-7.28 (m, 8H), 4.85 (d, J=5.5 Hz, 2H).

### Example 3: N-[(1-oxidopyridin-1-ium-2-yl)methyl]-3-[4-(trifluoromethoxy)phenyl]benzamide

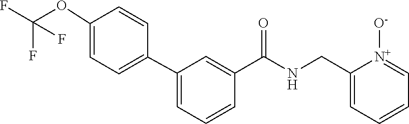 [SMILES: O=C(NCc1cccc[n+]1[O-])c1cccc(-c2ccc(OC(F)(F)F)cc2)c1]

The procedure given in Example 1 was followed using 4-trifluoromethoxyphenyl boronic acid as a reactant, instead of 4-trifluoromethylphenyl boronic acid, to give N-[(1-oxidopyridin-1-ium-2-yl)methyl]-3-[4-(trifluoromethoxy)phenyl]benzamide.

1H-NMR (CDCl₃, 500 MHz) δ=8.26 (d, J=6.5 Hz, 1H), 8.01 (s, 1H), 7.95 (brs, 1H, NH), 7.77 (d, J=7.5 Hz, 1H), 7.67 (d, J=7.5 Hz, 1H), 7.61-7.28 (m, 8H), 4.86 (d, J=6.0 Hz, 2H).

### Example 4: 3-(3-chlorophenyl)-N-[(1-oxidopyridin-1-ium-2-yl)methyl]benzamide

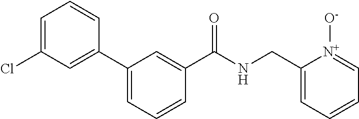 [SMILES: O=C(NCc1cccc[n+]1[O-])c1cccc(-c2cccc(Cl)c2)c1]

The procedure given in Example 1 was followed using 3-chlorophenyl boronic acid as a reactant, instead of 4-trifluoromethylphenyl boronic acid, to give 3-(3-chlorophenyl)-N-[(1-oxidopyridin-1-ium-2-yl)methyl]benzamide.

1H-NMR (CDCl₃, 500 MHz) δ=8.25-8.23 (m, 1H), 7.92 (s, 1H), 7.83 (brs, 1H, NH), 7.77-7.75 (m, 1H), 7.67 (d, J=7.5 Hz, 2H), 7.58-7.29 (m, 7H), 4.87 (s, 2H).

### Example 5: N-[(1-oxidopyridin-1-ium-2-yl)methyl]-3-[3-(trifluoromethyl)phenyl]benzamide

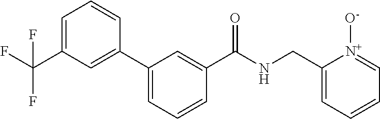 [SMILES: O=C(NCc1cccc[n+]1[O-])c1cccc(-c2cccc(C(F)(F)F)c2)c1]

The procedure given in Example 1 was followed using 3-trifluoromethylphenyl boronic acid as a reactant, instead of 4-trifluoromethylphenyl boronic acid, to give N-[(1-oxidopyridin-1-ium-2-yl)methyl]-3-[3-(trifluoromethyl)phenyl]benzamide.

1H-NMR (CDCl₃, 500 MHz) δ=8.23-8.14 (m, 1H), 8.06 (s, 1H), 7.95 (brs, 1H, NH), 7.78-7.46 (m, 8H), 7.30-7.28 (m, 2H), 4.87 (s, 2H).

### Example 6: 3-(3,4-dichlorophenyl)-N-[(1-oxidopyridin-1-ium-2-yl)methyl]benzamide

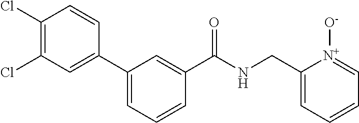 [SMILES: O=C(NCc1cccc[n+]1[O-])c1cccc(-c2ccc(Cl)c(Cl)c2)c1]

The procedure given in Example 1 was followed using 3,4-dichlorophenyl boronic acid as a reactant, instead of 4-trifluoromethylphenyl boronic acid, to give 3-(3,4-dichlorophenyl)-N-[(1-oxidopyridin-1-ium-2-yl)methyl]benzamide.

1H-NMR (CDCl₃, 500 MHz) δ=8.25 (d, J=6.5 Hz, 1H), 8.03 (s, 1H), 7.95 (brs, 1H, NH), 7.76 (d, J=7.5 Hz, 1H), 7.69-7.28 (m, 9H), 4.85 (d, J=6.5 Hz, 2H).

### Example 7: 3-(3,5-dichlorophenyl)-N-[(1-oxidopyridin-1-ium-2-yl)methyl]benzamide

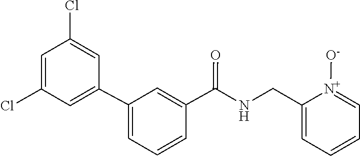 [SMILES: O=C(NCc1cccc[n+]1[O-])c1cccc(-c2cc(Cl)cc(Cl)c2)c1]

The procedure given in Example 1 was followed using 3,5-dichlorophenyl boronic acid as a reactant, instead of 4-trifluoromethylphenyl boronic acid, to give 3-(3,5-dichlorophenyl)-N-[(1-oxidopyridin-1-ium-2-yl)methyl]benzamide.

1H-NMR (CDCl₃, 500 MHz) δ=8.26 (d, J=6.0 Hz, 1H), 8.00 (s, 1H), 7.92 (brs, 1H, NH), 7.78 (d, J=8.0 Hz, 1H), 7.65 (t, J=2.5 Hz, 1H), 7.57 (dd, J=2.0, 7.5 Hz, 1H), 7.48-7.27 (m, 7H), 4.85 (d, J=6.0 Hz, 2H).

### Example 8: N-[(1-oxidopyridin-1-ium-2-yl)methyl]-3-[3-(trifluoromethoxy)phenyl]benzamide

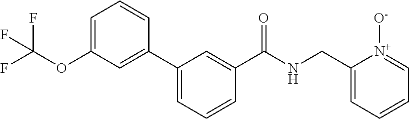 [SMILES: O=C(NCc1cccc[n+]1[O-])c1cccc(-c2cccc(OC(F)(F)F)c2)c1]

The procedure given in Example 1 was followed using 3-trifluoromethoxyphenyl boronic acid as a reactant, instead of 4-trifluoromethylphenyl boronic acid, to give N-[(1-oxidopyridin-1-ium-2-yl)methyl]-3-[3-(trifluoromethoxy)phenyl]benzamide.

1H-NMR (CDCl₃, 500 MHz) δ=8.24 (d, J=6.5 Hz, 1H), 7.99 (s, 1H), 7.79 (brs, 1H, NH), 7.78 (d, J=8.0 Hz, 1H), 7.67 (d, J=7.5 Hz, 1H), 7.57 (d, J=7.5 Hz, 1H), 7.52-7.20 (m, 7H), 4.85 (d, J=6.0 Hz, 2H).

### Example 9: 3-(2,4-dichlorophenyl)-N-[(1-oxidopyridin-1-ium-2-yl)methyl]benzamide

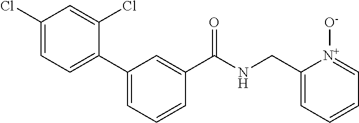 [SMILES: O=C(NCc1cccc[n+]1[O-])c1cccc(-c2ccc(Cl)cc2Cl)c1]

The procedure given in Example 1 was followed using 2,4-dichlorophenyl boronic acid as a reactant, instead of 4-trifluoromethylphenyl boronic acid, to give 3-(2,4-dichlorophenyl)-N-[(1-oxidopyridin-1-ium-2-yl)methyl]benzamide.

1H-NMR (CDCl₃, 500 MHz) δ=8.26 (d, J=6.0 Hz, 1H), 7.82 (brs, 1H, NH), 7.79 (dd, J=6.5, 8.0 Hz, 2H), 7.54 (dd, J=1.5, 7.5 Hz, 2H), 7.47 (dd, J=2.0, 7.5 Hz, 2H), 7.31-7.24 (m, 4H), 4.83 (d, J=6.0 Hz, 2H).

### Example 10: 3-(2,5-dichlorophenyl)-N-[(1-oxidopyridin-1-ium-2-yl)methyl]benzamide

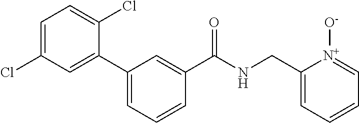 [SMILES: O=C(NCc1cccc[n+]1[O-])c1cccc(-c2cc(Cl)ccc2Cl)c1]

The procedure given in Example 1 was followed using 2,5-dichlorophenyl boronic acid as a reactant, instead of 4-trifluoromethylphenyl boronic acid, to give 3-(2,5-dichlorophenyl)-N-[(1-oxidopyridin-1-ium-2-yl)methyl]benzamide.

1H-NMR (CDCl₃, 500 MHz) δ=8.24 (d, J=6.5 Hz, 1H), 7.89 (brs, 1H, NH), 7.82-7.80 (m, 2H), 7.55-7.24 (m, 8H), 4.83 (d, J=6.0 Hz, 2H).

### Example 11: 3-[2,4-bis(trifluoromethyl)phenyl]-N-[(1-oxidopyridin-1-ium-2-yl)methyl]benzamide

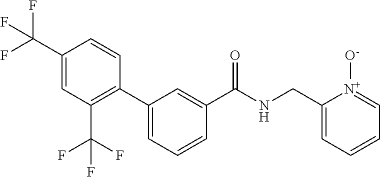 [SMILES: O=C(NCc1cccc[n+]1[O-])c1cccc(-c2ccc(C(F)(F)F)cc2C(F)(F)F)c1]

The procedure given in Example 1 was followed using 2,4-bis(trifluoromethyl)phenyl boronic acid as a reactant, instead of 4-trifluoromethylphenyl boronic acid, to give 3-[2,4-bis(trifluoromethyl)phenyl]-N-[(1-oxidopyridin-1-ium-2-yl)methyl]benzamide.

1H-NMR (CDCl₃, 500 MHz) δ=8.25 (d, J=6.0 Hz, 1H), 7.99 (s, 1H), 7.86 (brs, 1H, NH), 7.84-7.82 (m, 2H), 7.50 (s, 1H), 7.48-7.43 (m, 4H), 7.32-7.27 (m, 2H), 4.83 (d, J=6.0 Hz, 2H).

### Example 12: N-[(1-oxidopyridin-1-ium-2-yl)methyl]-3-phenyl-benzamide

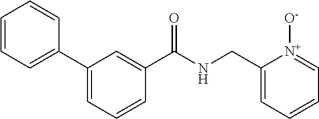 [SMILES: O=C(NCc1cccc[n+]1[O-])c1cccc(-c2ccccc2)c1]

The procedure given in Example 1 was followed using phenylboronic acid as a reactant, instead of 4-trifluoromethylphenyl boronic acid, to give N-[(1-oxidopyridin-1-ium-2-yl)methyl]-3-phenyl-benzamide.

1H-NMR (CDCl₃, 500 MHz) δ=8.24 (d, J=6.0 Hz, 1H), 7.03 (s, 1H), 7.95 (brs, 1H, NH), 7.75-7.29 (m, 11H), 4.84 (s, 2H).

### Example 13: 3-(4-tert-butylphenyl)-N-[(1-oxidopyridin-1-ium-2-yl)methyl]benzamide

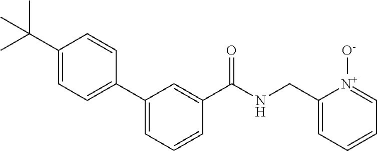 [SMILES: CC(C)(C)c1ccc(-c2cccc(C(=O)NCc3cccc[n+]3[O-])c2)cc1]

The procedure given in Example 1 was followed using 4-tert-butylphenyl boronic acid as a reactant, instead of 4-trifluoromethylphenyl boronic acid, to give 3-(4-tert-butylphenyl)-N-[(1-oxidopyridin-1-ium-2-yl)methyl]benzamide.

1H-NMR (CDCl₃, 500 MHz) δ=8.26 (d, J=1.0 Hz, 1H), 8.04 (s, 1H), 7.88 (brs, 1H, NH), 7.75-7.71 (m, 2H), 7.57-7.55 (m, 3H), 7.49-7.46 (m, 3H), 7.31-7.27 (m, 2H), 4.87 (d, J=6.5 Hz, 2H), 1.37 (s, 9H).

### Example 14: N-[(1-oxidopyridin-1-ium-2-yl)methyl]-3-(p-tolyl)benzamide

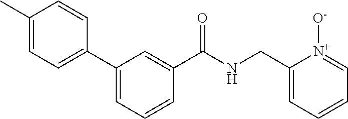 [SMILES: Cc1ccc(-c2cccc(C(=O)NCc3cccc[n+]3[O-])c2)cc1]

The procedure given in Example 1 was followed using p-tolyl boronic acid as a reactant, instead of 4-trifluoromethylphenyl boronic acid, to give N-[(1-oxidopyridin-1-ium-2-yl)methyl]-3-(p-tolyl)benzamide.

1H-NMR (CDCl₃, 500 MHz) δ=8.24 (d, J=6.0 Hz, 1H), 8.01 (s, 1H), 7.91 (brs, 1H, NH), 7.72 (d, J=7.5 Hz, 1H), 7.68 (d, J=8.0 Hz, 1H), 7.55 (d, J=7.5 Hz, 1H), 7.49-7.43 (m, 3H), 7.29-7.23 (m, 4H), 4.84 (d, J=6.0 Hz, 2H), 2.39 (s, 3H).

### Example 15: 3-(4-chloro-2-methyl-phenyl)-N-[(1-oxidopyridin-1-ium-2-yl)methyl]benzamide

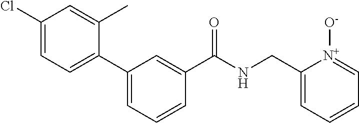 [SMILES: Cc1cc(Cl)ccc1-c1cccc(C(=O)NCc2cccc[n+]2[O-])c1]

The procedure given in Example 1 was followed using 4-chloro-2-methylphenyl boronic acid as a reactant, instead of 4-trifluoromethylphenyl boronic acid, to give 3-(4-chloro-2-methyl-phenyl)-N-[(1-oxidopyridin-1-ium-2-yl)methyl]benzamide.

1H-NMR (CDCl₃, 500 MHz) δ=8.24 (d, J=4.5 Hz, 1H), 7.90 (brs, 1H, NH), 7.76 (d, J=8.0 Hz, 1H), 7.71 (s, 1H), 7.55 (d, J=6.5 Hz, 1H), 7.46-7.27 (m, 5H), 7.20 (d, J=8.5 Hz, 1H), 7.11 (d, J=8.0 Hz, 1H), 4.83 (d, J=5.0 Hz, 2H), 2.20 (s, 3H).

### Example 16: 3-[2-fluoro-4-(trifluoromethyl)phenyl]-N-[(1-oxidopyridin-1-ium-2-yl)methyl]benzamide

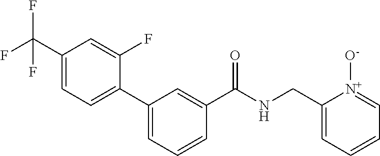 [SMILES: O=C(NCc1cccc[n+]1[O-])c1cccc(-c2ccc(C(F)(F)F)cc2F)c1]

The procedure given in Example 1 was followed using 2-fluoro-4-trifluoromethylphenyl boronic acid as a reactant, instead of 4-trifluoromethylphenyl boronic acid, to give 3-[2-fluoro-4-(trifluoromethyl)phenyl]-N-[(1-oxidopyridin-1-ium-2-yl)methyl]benzamide.

1H-NMR (CDCl₃, 500 MHz) δ=8.24 (d, J=5.5 Hz, 1H), 7.98 (s, 2H), 7.83 (d, J=7.5 Hz, 1H), 7.67 (d, J=7.5 Hz, 1H), 7.58-7.28 (m, 7H), 4.85 (d, J=5.5 Hz, 2H).

### Example 17: 3-(3-chloro-4-fluoro-phenyl)-N-[(1-oxidopyridin-1-ium-2-yl)methyl]benzamide

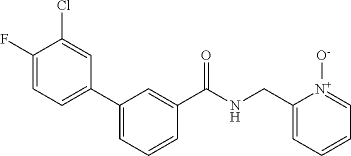 [SMILES: O=C(NCc1cccc[n+]1[O-])c1cccc(-c2ccc(F)c(Cl)c2)c1]

The procedure given in Example 1 was followed using 3-chloro-4-fluorophenyl boronic acid as a reactant, instead of 4-trifluoromethylphenyl boronic acid, to give 3-(3-chloro-4-fluoro-phenyl)-N-[(1-oxidopyridin-1-ium-2-yl)methyl]benzamide.

1H-NMR (CDCl₃, 500 MHz) δ=8.25 (d, J=6.5 Hz, 1H), 7.97 (s, 1H), 7.93 (brs, 1H, NH), 7.76 (d, J=8.0 Hz, 1H), 7.63-7.61 (m, 2H), 7.56 (d, J=7.5 Hz, 1H), 7.49-7.28 (m, 5H), 4.85 (d, J=6.0 Hz, 2H).

### Example 18: 3-(4-fluorophenyl)-N-[(1-oxidopyridin-1-ium-2-yl)methyl]benzamide

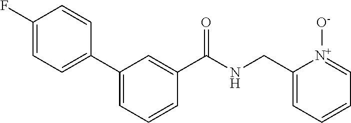 [SMILES: O=C(NCc1cccc[n+]1[O-])c1cccc(-c2ccc(F)cc2)c1]

The procedure given in Example 1 was followed using 4-fluorophenyl boronic acid as a reactant, instead of 4-trifluoromethylphenyl boronic acid, to give 3-(4-fluorophenyl)-N-[(1-oxidopyridin-1-ium-2-yl)methyl]benzamide.

1H-NMR (CDCl₃, 500 MHz) δ=8.25 (d, J=6.0 Hz, 1H), 7.98 (s, 1H), 7.91 (brs, 1H, NH), 7.74 (d, J=7.5 Hz, 1H), 7.65 (d, J=7.0 Hz, 1H), 7.56-7.54 (m, 3H), 7.47 (t, J=7.5 Hz, 1H), 7.31-7.26 (m, 2H), 7.12 (t, J=8.5 Hz, 2H), 4.85 (d, J=4.5 Hz, 2H).

### Example 19: 3-(3,4-difluorophenyl)-N-[(1-oxidopyridin-1-ium-2-yl)methyl]benzamide

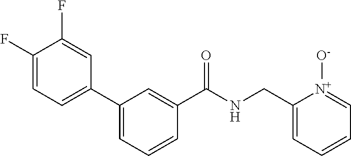 [SMILES: O=C(NCc1cccc[n+]1[O-])c1cccc(-c2ccc(F)c(F)c2)c1]

The procedure given in Example 1 was followed using 3,4-difluorophenyl boronic acid as a reactant, instead of 4-trifluoromethylphenyl boronic acid, to give 3-(3,4-difluorophenyl)-N-[(1-oxidopyridin-1-ium-2-yl)methyl]benzamide.

1H-NMR (CDCl₃, 500 MHz) δ=8.25 (d, J=6.0 Hz, 1H), 7.97 (s, 1H), 7.94 (brs, 1H, NH), 7.76 (d, J=7.5 Hz, 1H), 7.63 (d, J=8.0 Hz, 1H), 7.57-7.56 (m, 1H), 7.49-7.46 (m, 1H), 7.41-7.28 (m, 4H), 7.21 (t, J=10.0 Hz, 1H), 4.85 (d, J=6.0 Hz, 2H).

### Example 20: 3-(2-chlorophenyl)-N-[(1-oxidopyridin-1-ium-2-yl)methyl]benzamide

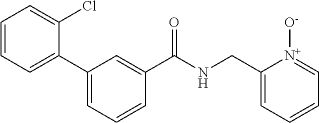 [SMILES: O=C(NCc1cccc[n+]1[O-])c1cccc(-c2ccccc2Cl)c1]

The procedure given in Example 1 was followed using 2-chlorophenyl boronic acid as a reactant, instead of 4-trifluoromethylphenyl boronic acid, to give 3-(2-chlorophenyl)-N-[(1-oxidopyridin-1-ium-2-yl)methyl]benzamide.

1H-NMR (CDCl₃, 500 MHz) δ=8.25 (d, J=6.5 Hz, 1H), 7.87 (brs, 1H, NH), 7.85 (s, 1H), 7.82 (d, J=7.5 Hz, 1H), 7.59-7.28 (m, 9H), 4.85 (d, J=6.0 Hz, 2H).

### Example 21: N-[(1-oxidopyridin-1-ium-2-yl)methyl]-3-[2-(trifluoromethyl)phenyl]benzamide

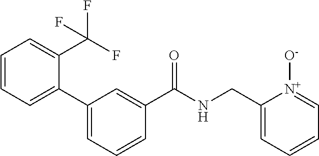 [SMILES: O=C(NCc1cccc[n+]1[O-])c1cccc(-c2ccccc2C(F)(F)F)c1]

The procedure given in Example 1 was followed using 2-trifluoromethylphenyl boronic acid as a reactant, instead of 4-trifluoromethylphenyl boronic acid, to give N-[(1-oxidopyridin-1-ium-2-yl)methyl]-3-[2-(trifluoromethyl)phenyl]benzamide.

1H-NMR (CDCl₃, 500 MHz) δ=8.25 (d, J=6.0 Hz, 1H), 7.88 (brs, 1H, NH), 7.84-7.74 (m, 3H), 7.57-7.43 (m, 5H), 7.33-7.28 (m, 3H), 4.84 (d, J=6.5 Hz, 2H).

### Example 22: 3-(4-fluoro-3-methyl-phenyl)-N-[(1-oxidopyridin-1-ium-2-yl)methyl]benzamide

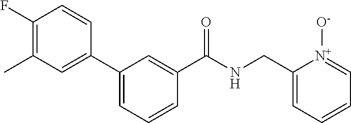 [SMILES: Cc1cc(-c2cccc(C(=O)NCc3cccc[n+]3[O-])c2)ccc1F]

The procedure given in Example 1 was followed using 4-fluoro-3-methylphenyl boronic acid as a reactant, instead of 4-trifluoromethylphenyl boronic acid, to give 3-(4-fluoro-3-methyl-phenyl)-N-[(1-oxidopyridin-1-ium-2-yl)methyl]benzamide.

1H-NMR (CDCl₃, 500 MHz) δ=8.26 (d, J=6.0 Hz, 1H), 7.98 (s, 1H), 7.91 (brs, 1H, NH), 7.73 (d, J=7.5 Hz, 1H), 7.65 (d, J=7.5 Hz, 1H), 7.59 (d, J=6.5 Hz, 1H), 7.47-7.28 (m, 5H), 7.06 (t, J=9.0 Hz, 1H), 4.86 (d, J=6.0 Hz, 2H), 2.33 (s, 3H).

### Example 23: 3-(m-tolyl)-N-[(1-oxidopyridin-1-ium-2-yl)methyl]benzamide

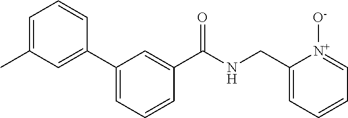 [SMILES: Cc1cccc(-c2cccc(C(=O)NCc3cccc[n+]3[O-])c2)c1]

The procedure given in Example 1 was followed using 3-methylphenyl boronic acid as a reactant, instead of 4-trifluoromethylphenyl boronic acid, to give 3-(m-tolyl)-N-[(1-oxidopyridin-1-ium-2-yl)methyl]benzamide.

1H-NMR (CDCl₃, 500 MHz) δ=8.24 (d, J=6.0 Hz, 1H), 8.02 (brs, 1H, NH), 7.90 (s, 1H), 7.74 (d, J=8.0 Hz, 1H), 7.69 (d, J=7.5 Hz, 1H), 7.55 (dd, J=2.0, 7.5 Hz, 1H), 7.47-7.28 (m, 6H), 7.17 (d, J=7.5 Hz, 1H), 4.85 (d, J=6.0 Hz, 2H), 2.41 (s, 3H).

### Example 24: 3-(4-isopropylphenyl)-N-[(1-oxidopyridin-1-ium-2-yl)methyl]benzamide

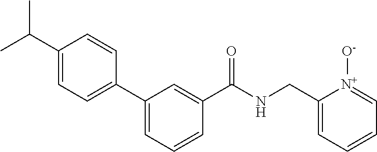 [SMILES: CC(C)c1ccc(-c2cccc(C(=O)NCc3cccc[n+]3[O-])c2)cc1]

The procedure given in Example 1 was followed using 4-isopropylphenyl boronic acid as a reactant, instead of 4-trifluoromethylphenyl boronic acid, to give 3-(4-isopropylphenyl)-N-[(1-oxidopyridin-1-ium-2-yl)methyl]benzamide.

1H-NMR (CDCl₃, 500 MHz) δ=8.27 (d, J=6.5 Hz, 1H), 8.03 (s, 1H), 7.91 (brs, 1H, NH), 7.75 (d, J=8.0 Hz, 1H), 7.71 (d, J=7.5 Hz, 1H), 7.58-7.54 (m, 3H), 7.47 (t, J=8.0 Hz, 1H), 7.34-7.27 (m, 4H), 4.87 (d, J=6.0 Hz, 2H), 2.99-2.94 (m, 1H), 1.30 (d, J=7.0 Hz, 6H).

### Example 25: 3-(2-fluorophenyl)-N-[(1-oxidopyridin-1-ium-2-yl)methyl]benzamide

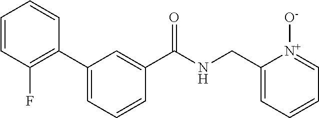 [SMILES: O=C(NCc1cccc[n+]1[O-])c1cccc(-c2ccccc2F)c1]

The procedure given in Example 1 was followed using 2-fluorophenylboronic acid as a reactant, instead of 4-trifluoromethylphenyl boronic acid, to give 3-(2-fluorophenyl)-N-[(1-oxidopyridin-1-ium-2-yl)methyl]benzamide.

1H-NMR (CDCl₃, 500 MHz) δ=8.08 (s, 1H), 7.82 (s, 1H), 7.74 (d, J=5.0 Hz, 1H), 7.51-7.30 (m, 8H), 7.24-7.06 (m, 2H), 4.85 (s, 2H).

### Example 26: 3-(4-chloro-2-methoxy-phenyl)-N-[(1-oxidopyridin-1-ium-2-yl)methyl]benzamide

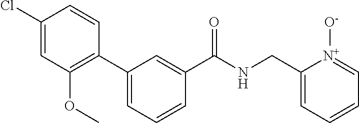 [SMILES: COc1cc(Cl)ccc1-c1cccc(C(=O)NCc2cccc[n+]2[O-])c1]

The procedure given in Example 1 was followed using 4-chloro-2-methoxyphenylboronic acid as a reactant, instead of 4-trifluoromethylphenyl boronic acid, to give 3-(4-chloro-2-methoxy-phenyl)-N-[(1-oxidopyridin-1-ium-2-yl)methyl]benzamide.

1H-NMR (CDCl₃, 500 MHz) δ=8.26 (d, J=6.5 Hz, 1H), 7.92 (s, 1H), 7.89 (brs, 1H, NH), 7.77 (d, J=8.0 Hz, 1H), 7.61 (d, J=7.5 Hz, 1H), 7.57 (dd, J=1.5, 7.5 Hz, 1H), 7.45 (t, J=8.0 Hz, 1H), 7.33-7.26 (m, 2H), 7.23 (d, J=8.0 Hz, 1H), 7.02 (dd, J=2.0, 8.0 Hz, 1H), 6.97 (d, J=2.0 Hz, 1H), 4.86 (d, J=6.0 Hz, 2H), 3.81 (s, 3H).

### Example 27: 3-[4-chloro-2-(trifluoromethyl)phenyl]-N-[(1-oxidopyridin-1-ium-2-yl)methyl]benzamide

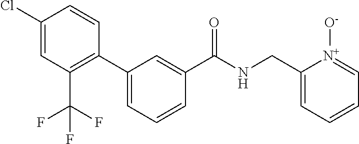 [SMILES: O=C(NCc1cccc[n+]1[O-])c1cccc(-c2ccc(Cl)cc2C(F)(F)F)c1]

The procedure given in Example 1 was followed using 4-chloro-2-trifluoromethylphenyl boronic acid as a reactant, instead of 4-trifluoromethylphenyl boronic acid, to give 3-[4-chloro-2-(trifluoromethyl)phenyl]-N-[(1-oxidopyridin-1-ium-2-yl)methyl]benzam ide.

1H-NMR (CDCl₃, 500 MHz) δ=8.24 (dd, J=1.5, 7.5 Hz, 1H), 7.90 (brs, 1H, NH), 7.84-7.81 (m, 1H), 7.73 (d, J=2.5 Hz, 1H), 7.55 (d, J=2.0 Hz, 1H), 7.47-7.41 (m, 2H), 7.33-7.24 (m, 5H), 4.83 (d, J=7.5 Hz, 2H).

### Example 28: 3-(4-chloro-3-methyl-phenyl)-N-[(1-oxidopyridin-1-ium-2-yl)methyl]benzamide

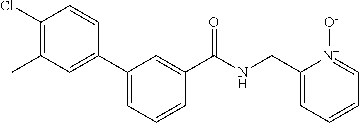 [SMILES: Cc1cc(-c2cccc(C(=O)NCc3cccc[n+]3[O-])c2)ccc1Cl]

The procedure given in Example 1 was followed using 4-chloro-3-methylphenyl boronic acid as a reactant, instead of 4-trifluoromethylphenyl boronic acid, to give 3-(4-chloro-3-methyl-phenyl)-N-[(1-oxidopyridin-1-ium-2-yl)methyl]benzamide.

1H-NMR (CDCl₃, 500 MHz) δ=8.26 (s, 1H), 8.02 (s, 1H), 7.93 (brs, 1H, NH), 7.76 (d, J=6.5 Hz, 1H), 7.68 (d, J=7.5 Hz, 1H), 7.61 (s, 1H), 7.49-7.30 (m, 6H), 4.88 (s, 2H), 2.44 (s, 3H).

### Example 29: 3-(3-chloro-5-fluoro-phenyl)-N-[(1-oxidopyridin-1-ium-2-yl)methyl]benzamide

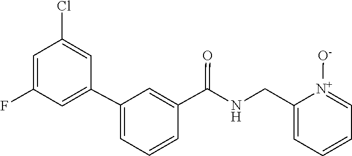 [SMILES: O=C(NCc1cccc[n+]1[O-])c1cccc(-c2cc(F)cc(Cl)c2)c1]

The procedure given in Example 1 was followed using 3-chloro-5-fluorophenyl boronic acid as a reactant, instead of 4-trifluoromethylphenyl boronic acid, to give 3-(3-chloro-5-fluoro-phenyl)-N-[(1-oxidopyridin-1-ium-2-yl)methyl]benzamide.

1H-NMR (CDCl₃, 500 MHz) δ=8.25 (d, J=6.0 Hz, 1H), 7.99 (s, 1H), 7.92 (brs, 1H, NH), 7.78 (d, J=7.5 Hz, 1H), 7.64 (d, J=7.5 Hz, 1H), 7.57-7.47 (m, 2H), 7.37 (s, 1H), 7.33-7.27 (m, 2H), 7.19 (d, J=9.5 Hz, 1H), 7.08 (d, J=8.5 Hz, 1H), 4.85 (d, J=6.0 Hz, 2H).

### Example 30: 3-(4-chloro-3-fluoro-phenyl)-N-[(1-oxidopyridin-1-ium-2-yl)methyl]benzamide

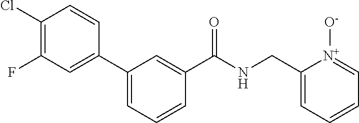 [SMILES: O=C(NCc1cccc[n+]1[O-])c1cccc(-c2ccc(Cl)c(F)c2)c1]

The procedure given in Example 1 was followed using 4-chloro-3-fluorophenyl boronic acid as a reactant, instead of 4-trifluoromethylphenyl boronic acid, to give 3-(4-chloro-3-fluoro-phenyl)-N-[(1-oxidopyridin-1-ium-2-yl)methyl]benzamide.

1H-NMR (CDCl₃, 500 MHz) δ=8.29 (d, J=6.0 Hz, 1H), 8.03 (s, 1H), 7.97 (brs, 1H, NH), 7.78 (d, J=8.0 Hz, 1H), 7.67 (d, J=1.0 Hz, 1H), 7.65 (dd, J=5.0, 6.0 Hz, 1H), 7.60 (dd, J=2.0, 8.0 Hz, 1H), 7.49-7.28 (m, 5H), 4.87 (d, J=6.0 Hz, 2H).

### Example 31: 3-(2,4-difluorophenyl)-N-[(1-oxidopyridin-1-ium-2-yl)methyl]benzamide

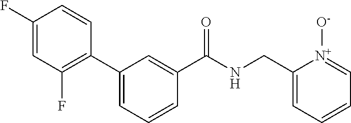 [SMILES: O=C(NCc1cccc[n+]1[O-])c1cccc(-c2ccc(F)cc2F)c1]

The procedure given in Example 1 was followed using 2,4-difluorophenyl boronic acid as a reactant, instead of 4-trifluoromethylphenyl boronic acid, to give 3-(2,4-difluorophenyl)-N-[(1-oxidopyridin-1-ium-2-yl)methyl]benzamide.

1H-NMR (CDCl₃, 500 MHz) δ=8.25 (d, J=6.0 Hz, 1H), 7.92 (s, 1H), 7.90 (brs, 1H, NH), 7.78 (d, J=7.5 Hz, 1H), 7.63 (d, J=7.0 Hz, 1H), 7.56 (dd, J=1.0, 6.0 Hz, 1H), 7.49 (t, J=7.5 Hz, 1H), 7.43-7.40 (m, 1H), 7.38-7.27 (m, 2H), 6.98-6.89 (m, 2H), 4.86 (d, J=6.0 Hz, 2H).

### Example 32: 3-(3,5-difluorophenyl)-N-[(1-oxidopyridin-1-ium-2-yl)methyl]benzamide

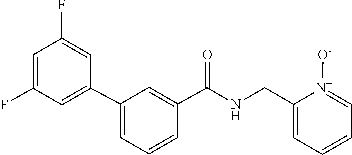 [SMILES: O=C(NCc1cccc[n+]1[O-])c1cccc(-c2cc(F)cc(F)c2)c1]

The procedure given in Example 1 was followed using 3,5-difluorophenyl boronic acid as a reactant, instead of 4-trifluoromethylphenyl boronic acid, to give 3-(3,5-difluorophenyl)-N-[(1-oxidopyridin-1-ium-2-yl)methyl]benzamide.

1H-NMR (CDCl₃, 500 MHz) δ=8.27 (d, J=6.0 Hz, 1H), 7.99 (s, 1H), 7.94 (brs, 1H, NH), 7.78 (d, J=7.5 Hz, 1H), 7.66-7.27 (m, 5H), 7.13-7.10 (m, 2H), 6.82-6.78 (m, 1H), 4.86 (d, J=6.0 Hz, 2H).

### Example 33: N-[(1-oxidopyridin-1-ium-2-yl)methyl]-3-(3,4,5-trifluorophenyl)benzamide

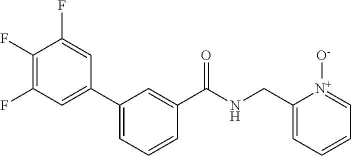 [SMILES: O=C(NCc1cccc[n+]1[O-])c1cccc(-c2cc(F)c(F)c(F)c2)c1]

The procedure given in Example 1 was followed using 3,4,5-trifluorophenyl boronic acid as a reactant, instead of 4-trifluoromethylphenyl boronic acid, to give N-[(1-oxidopyridin-1-ium-2-yl)methyl]-3-(3,4,5-trifluorophenyl)benzamide.

1H-NMR (CDCl₃, 500 MHz) δ=8.31 (d, J=6.0 Hz, 1H), 7.95 (s, 1H), 7.87 (brs, 1H, NH), 7.76 (d, J=8.0 Hz, 1H), 7.64 (d, J=1.5 Hz, 1H), 7.62-7.33 (m, 4H), 7.22-7.17 (m, 2H), 4.91 (d, J=6.0 Hz, 2H).

### Example 34: 3-(2,3-difluorophenyl)-N-[(1-oxidopyridin-1-ium-2-yl)methyl]benzamide

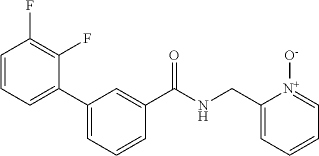 [SMILES: O=C(NCc1cccc[n+]1[O-])c1cccc(-c2cccc(F)c2F)c1]

The procedure given in Example 1 was followed using 2,3-difluorophenyl boronic acid as a reactant, instead of 4-trifluoromethylphenyl boronic acid, to give 3-(2,3-difluorophenyl)-N-[(1-oxidopyridin-1-ium-2-yl)methyl]benzamide.

1H-NMR (CDCl₃, 500 MHz) δ=8.27 (d, J=6.0 Hz, 1H), 7.97 (s, 1H), 7.95 (brs, 1H, NH), 7.83 (d, J=8.0 Hz, 1H), 7.68 (d, J=7.5 Hz, 1H), 7.58 (d, J=7.5 Hz, 1H), 7.52 (t, J=7.5 Hz, 1H), 7.35-7.27 (m, 2H), 7.21-7.14 (m, 3H), 4.87 (d, J=6.0 Hz, 2H).

### Example 35: 3-(3-isopropylphenyl)-N-[(1-oxidopyridin-1-ium-2-yl)methyl]benzamide

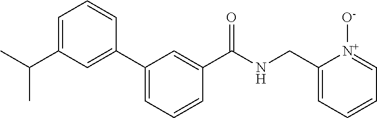 [SMILES: CC(C)c1cccc(-c2cccc(C(=O)NCc3cccc[n+]3[O-])c2)c1]

The procedure given in Example 1 was followed using 3-isopropylphenyl boronic acid as a reactant, instead of 4-trifluoromethylphenyl boronic acid, to give 3-(3-isopropylphenyl)-N-[(1-oxidopyridin-1-ium-2-yl)methyl]benzamide.

1H-NMR (CDCl₃, 500 MHz) δ=8.25 (d, J=6.5 Hz, 1H), 8.03 (s, 1H), 7.89 (brs, 1H, NH), 7.71 (dd, J=7.5, 14.0 Hz, 2H), 7.56 (d, J=7.5 Hz, 1H), 7.48-7.29 (m, 7H), 4.85 (d, J=6.0 Hz, 2H), 2.99-2.94 (m, 1H), 1.29 (d, J=6.5 Hz, 6H).

### Example 36: 3-(4-cyclopropylphenyl)-N-[(1-oxidopyridin-1-ium-2-yl)methyl]benzamide

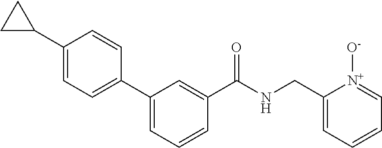 [SMILES: O=C(NCc1cccc[n+]1[O-])c1cccc(-c2ccc(C3CC3)cc2)c1]

The procedure given in Example 1 was followed using 4-cyclopropylphenyl boronic acid as a reactant, instead of 4-trifluoromethylphenyl boronic acid, to give 3-(4-cyclopropylphenyl)-N-[(1-oxidopyridin-1-ium-2-yl)methyl]benzamide.

1H-NMR (CDCl₃, 500 MHz) δ=8.25 (d, J=6.5 Hz, 1H), 8.02 (s, 1H), 7.93 (brs, 1H, NH), 7.74-7.68 (m, 2H), 7.57 (dd, J=2.0, 8.0 Hz, 1H), 7.51-7.45 (m, 3H), 7.33-7.26 (m, 2H), 7.15 (d, J=8.0 Hz, 2H), 4.86 (d, J=6.0 Hz, 2H), 1.97-1.92 (m, 1H), 1.03-0.98 (m, 2H), 0.77-0.75 (m, 2H).

### Example 37: 3-[4-(difluoromethoxy)phenyl]-N-[(1-oxidopyridin-1-ium-2-yl)methyl]benzamide

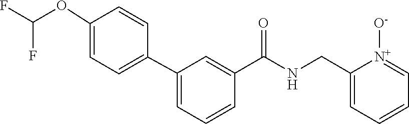 [SMILES: O=C(NCc1cccc[n+]1[O-])c1cccc(-c2ccc(OC(F)F)cc2)c1]

The procedure given in Example 1 was followed using 4-difluoromethoxyphenyl boronic acid as a reactant, instead of 4-trifluoromethylphenyl boronic acid, to give 3-[4-(difluoromethoxy)phenyl]-N-[(1-oxidopyridin-1-ium-2-yl)methyl]benzamide.

1H-NMR (CDCl₃, 500 MHz) δ=8.26 (d, J=6.5 Hz, 1H), 8.02 (s, 1H), 7.97 (brs, 1H, NH), 7.78-7.51 (m, 5H), 7.34-7.29 (m, 3H), 7.22 (d, J=8.5 Hz, 2H), 6.58 (d, J=74.0 Hz, 1H), 4.88 (d, J=6.0 Hz, 2H).

### Example 38: 3-[2-methyl-4-(trifluoromethyl)phenyl]-N-[(1-oxidopyridin-1-ium-2-yl)methyl]benzamide

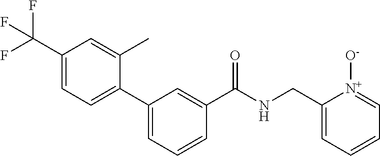 [SMILES: Cc1cc(C(F)(F)F)ccc1-c1cccc(C(=O)NCc2cccc[n+]2[O-])c1]

The procedure given in Example 1 was followed using 2-methyl-4-(trifluoromethyl)phenyl boronic acid as a reactant, instead of 4-trifluoromethylphenyl boronic acid, to give 3-[2-methyl-4-(trifluoromethyl)phenyl]-N-[(1-oxidopyridin-1-ium-2-yl)methyl]benzamide.

1H-NMR (CDCl₃, 500 MHz) δ=8.25 (d, J=6.5 Hz, 1H), 7.97 (brs, 1H, NH), 7.83 (d, J=7.5 Hz, 1H), 7.75 (s, 1H), 7.53-7.27 (m, 8H), 4.85 (d, J=6.5 Hz, 2H), 2.29 (s, 3H).

### Example 39: 3-[4-fluoro-3-(trifluoromethyl)phenyl]-N-[(1-oxidopyridin-1-ium-2-yl)methyl]benzamide

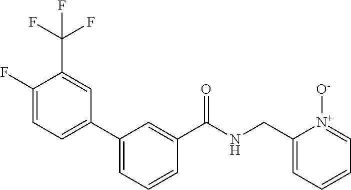 [SMILES: O=C(NCc1cccc[n+]1[O-])c1cccc(-c2ccc(F)c(C(F)(F)F)c2)c1]

The procedure given in Example 1 was followed using 4-fluoro-3-trifluoromethylphenyl boronic acid as a reactant, instead of 4-trifluoromethylphenyl boronic acid, to give 3-[4-fluoro-3-(trifluoromethyl)phenyl]-N-[(1-oxidopyridin-1-ium-2-yl)methyl]benzamide.

1H-NMR (CDCl₃, 500 MHz) δ=8.25 (d, J=6.5 Hz, 1H), 8.03 (s, 1H), 7.89 (brs, 1H, NH), 7.84-7.33 (m, 9H), 4.90 (d, J=6.5 Hz, 2H).

### Example 40: 3-(2-methoxyphenyl)-N-[(1-oxidopyridin-1-ium-2-yl)methyl]benzamide

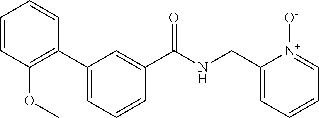 [SMILES: COc1ccccc1-c1cccc(C(=O)NCc2cccc[n+]2[O-])c1]

The procedure given in Example 1 was followed using 2-methoxyphenyl boronic acid as a reactant, instead of 4-trifluoromethylphenyl boronic acid, to give 3-(2-methoxyphenyl)-N-[(1-oxidopyridin-1-ium-2-yl)methyl]benzamide.

1H-NMR (CDCl₃, 500 MHz) δ=8.31 (d, J=6.0 Hz, 1H), 7.95 (s, 1H), 7.87 (brs, 1H, NH), 7.79 (d, J=7.5 Hz, 1H), 7.70 (d, J=8.0 Hz, 1H), 7.59 (d, J=7.0 Hz, 1H), 7.49 (t, J=7.5 Hz, 1H), 7.40-7.30 (m, 4H), 7.07 (t, J=7.0 Hz, 1H), 7.03 (d, J=8.0 Hz, 1H), 4.89 (d, J=5.0 Hz, 2H), 3.85 (s, 3H).

### Example 41: 3-[3-fluoro-5-(trifluoromethyl)phenyl]-N-[(1-oxidopyridin-1-ium-2-yl)methyl]benzamide

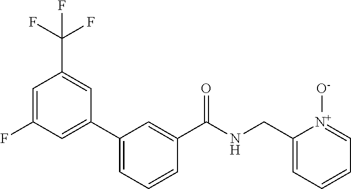 [SMILES: O=C(NCc1cccc[n+]1[O-])c1cccc(-c2cc(F)cc(C(F)(F)F)c2)c1]

The procedure given in Example 1 was followed using 3-fluoro-5-trifluoromethylphenyl boronic acid as a reactant, instead of 4-trifluoromethylphenyl boronic acid, to give 3-[3-fluoro-5-(trifluoromethyl)phenyl]-N-[(1-oxidopyridin-1-ium-2-yl)methyl]benzamide.

1H-NMR (CDCl₃, 500 MHz) δ=8.36 (d, J=6.0 Hz, 1H), 8.11 (s, 1H), 8.04 (brs, 1H, NH), 7.83 (d, J=7.5 Hz, 1H), 7.74 (d, J=8.0 Hz, 1H), 7.68 (s, 1H), 7.61-7.52 (m, 4H), 7.38-7.30 (m, 2H), 4.89 (d, J=6.0 Hz, 2H).

### Example 42: 3-[3-fluoro-4-(trifluoromethyl)phenyl]-N-[(1-oxidopyridin-1-ium-2-yl)methyl]benzamide

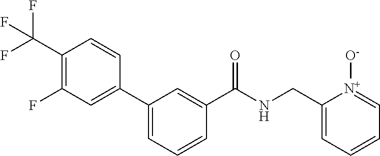 [SMILES: O=C(NCc1cccc[n+]1[O-])c1cccc(-c2ccc(C(F)(F)F)c(F)c2)c1]

The procedure given in Example 1 was followed using 3-fluoro-4-trifluoromethylphenyl boronic acid as a reactant, instead of 4-trifluoromethylphenyl boronic acid, to give 3-[3-fluoro-4-(trifluoromethyl)phenyl]-N-[(1-oxidopyridin-1-ium-2-yl)methyl]benzamide.

1H-NMR (CDCl₃, 500 MHz) δ=8.31 (d, J=6.0 Hz, 1H), 8.19 (s, 1H), 7.99 (brs, 1H, NH), 7.83 (d, J=7.5 Hz, 1H), 7.72-7.30 (m, 8H), 4.88 (d, J=6.0 Hz, 2H).

### Example 43: 3-[3-methyl-4-(trifluoromethyl)phenyl]-N-[(1-oxidopyridin-1-ium-2-yl)methyl]benzamide

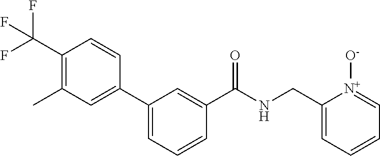 [SMILES: Cc1cc(-c2cccc(C(=O)NCc3cccc[n+]3[O-])c2)ccc1C(F)(F)F]

The procedure given in Example 1 was followed using 3-methyl-4-trifluoromethylphenyl boronic acid as a reactant, instead of 4-trifluoromethylphenyl boronic acid, to give 3-[3-methyl-4-(trifluoromethyl)phenyl]-N-[(1-oxidopyridin-1-ium-2-yl)methyl]benzamide.

1H-NMR (CDCl₃, 500 MHz) δ=8.39 (d, J=6.0 Hz, 1H), 8.08 (s, 1H), 7.94 (brs, 1H, NH), 7.80 (d, J=7.5 Hz, 1H), 7.73-7.29 (m, 8H), 4.88 (d, J=6.0 Hz, 2H), 2.57 (s, 3H).

### Example 44: 3-[4-(1-hydroxy-1-methyl-ethyl)phenyl]-N-[(1-oxidopyridin-1-ium-2-yl)methyl]benzamide

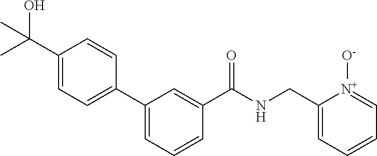 [SMILES: CC(C)(O)c1ccc(-c2cccc(C(=O)NCc3cccc[n+]3[O-])c2)cc1]

The procedure given in Example 1 was followed using 1-hydroxy-1-methyl-ethylphenyl boronic acid as a reactant, instead of 4-trifluoromethylphenyl boronic acid, to give 3-[4-(1-hydroxy-1-methyl-ethyl)phenyl]-N-[(1-oxidopyridin-1-ium-2-yl)methyl]benzamide.

1H-NMR (CDCl₃, 500 MHz) δ=8.26 (d, J=6.0 Hz, 1H), 8.05 (s, 1H), 7.92 (brs, 1H, NH), 7.76 (d, J=7.5 Hz, 1H), 7.73 (d, J=8.0 Hz, 1H), 7.61-7.28 (m, 8H), 4.88 (d, J=6.0 Hz, 2H), 1.64 (s, 6H).

### Example 45: 3-[3-methoxy-5-(trifluoromethyl)phenyl]-N-[(1-oxidopyridin-1-ium-2-yl)methyl]benzamide

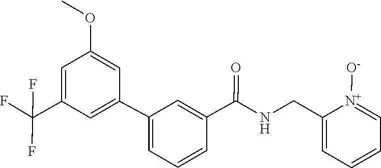 [SMILES: COc1cc(-c2cccc(C(=O)NCc3cccc[n+]3[O-])c2)cc(C(F)(F)F)c1]

The procedure given in Example 1 was followed using 3-methoxy-5-trifluoromethylphenyl boronic acid as a reactant, instead of 4-trifluoromethylphenyl boronic acid, to give 3-[3-methoxy-5-(trifluoromethyl)phenyl]-N-[(1-oxidopyridin-1-ium-2-yl)methyl]benzamide.

1H-NMR (CDCl₃, 500 MHz) δ=8.27 (d, J=6.0 Hz, 1H), 8.06 (s, 1H), 7.92 (brs, 1H, NH), 7.79 (d, J=7.5 Hz, 1H), 7.72 (d, J=8.0 Hz, 1H), 7.59-7.30 (m, 6H), 7.14 (s, 1H), 4.88 (d, J=6.0 Hz, 2H), 3.92 (s, 3H).

### Example 46: 3-[2-methoxy-4-(trifluoromethoxy)phenyl]-N-[(1-oxidopyridin-1-ium-2-yl)methyl]benzamide

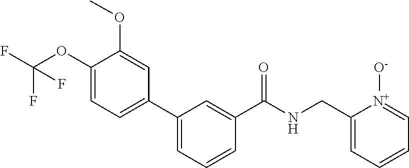 [SMILES: COc1cc(-c2cccc(C(=O)NCc3cccc[n+]3[O-])c2)ccc1OC(F)(F)F]

The procedure given in Example 1 was followed using 2-methoxy-4-trifluoromethoxyphenyl boronic acid as a reactant, instead of 4-trifluoromethylphenyl boronic acid, to give 3-[2-methoxy-4-(trifluoromethoxy)phenyl]-N-[(1-oxidopyridin-1-ium-2-yl)methyl]benzamide.

1H-NMR (CDCl₃, 500 MHz) δ=8.24 (d, J=6.0 Hz, 1H), 7.91 (s, 1H), 7.86 (brs, 1H, NH), 7.77 (d, J=7.5 Hz, 1H), 7.61 (d, J=8.0 Hz, 1H), 7.55-7.28 (m, 5H), 6.90 (d, J=7.0 Hz, 1H), 6.82 (s, 1H), 7.59-7.30 (m, 6H), 7.14 (s, 1H), 4.85 (d, J=6.0 Hz, 2H), 3.82 (s, 3H).

### Example 47: 3-(4,5-difluoro-2-methoxy-phenyl)-N-[(1-oxidopyridin-1-ium-2-yl)methyl]benzamide

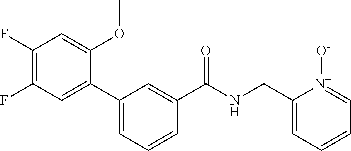 [SMILES: COc1cc(F)c(F)cc1-c1cccc(C(=O)NCc2cccc[n+]2[O-])c1]

The procedure given in Example 1 was followed using 4,5-difluoro-2-methoxyphenyl boronic acid as a reactant, instead of 4-trifluoromethylphenyl boronic acid, to give 3-(4,5-difluoro-2-methoxy-phenyl)-N-[(1-oxidopyridin-1-ium-2-yl)methyl]benzamide.

1H-NMR (CDCl₃, 500 MHz) δ=8.25 (d, J=6.0 Hz, 1H), 7.89 (s, 1H), 7.88 (brs, 1H, NH), 7.76 (d, J=2.5 Hz, 1H), 7.58-7.54 (m, 2H), 7.45 (t, J=7.5 Hz, 1H), 7.30-7.28 (m, 2H), 7.13 (t, J=10.0 Hz, 1H), 6.79 (dd, J=7.0, 12.0 Hz, 1H), 4.84 (d, J=6.0 Hz, 2H), 3.76 (s, 3H).

### Example 48: 3-(4-chloro-3-methoxy-phenyl)-N-[(1-oxidopyridin-1-ium-2-yl)methyl]benzamide

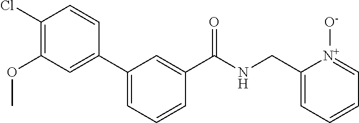 [SMILES: COc1cc(-c2cccc(C(=O)NCc3cccc[n+]3[O-])c2)ccc1Cl]

The procedure given in Example 1 was followed using 4-chloro-3-methoxyphenyl boronic acid as a reactant, instead of 4-trifluoromethylphenyl boronic acid, to give 3-(4-chloro-3-methoxy-phenyl)-N-[(1-oxidopyridin-1-ium-2-yl)methyl]benzamide.

1H-NMR (CDCl₃, 500 MHz) δ=8.25 (d, J=6.0 Hz, 1H), 8.01 (s, 1H), 7.91 (brs, 1H, NH), 7.75-7.73 (m, 1H), 7.68-7.66 (m, 1H), 7.56-7.50 (m, 1H), 7.48-7.41 (m, 2H), 7.31-7.27 (m, 2H), 7.12-7.10 (m, 2H), 4.85 (d, J=6.0 Hz, 2H), 3.79 (s, 3H).

### Example 49: 3-[3-methoxy-4-(trifluoromethyl)phenyl]-N-[(1-oxidopyridin-1-ium-2-yl)methyl]benzamide

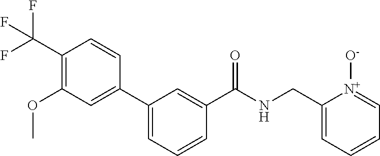 [SMILES: COc1cc(-c2cccc(C(=O)NCc3cccc[n+]3[O-])c2)ccc1C(F)(F)F]

The procedure given in Example 1 was followed using 3-methoxy-4-trifluoromethylphenyl boronic acid as a reactant, instead of 4-trifluoromethylphenyl boronic acid, to give 3-[3-methoxy-4-(trifluoromethyl)phenyl]-N-[(1-oxidopyridin-1-ium-2-yl)methyl]benzamide.

1H-NMR (CDCl₃, 500 MHz) δ=8.24 (d, J=6.0 Hz, 1H), 8.03 (s, 1H), 7.96 (brs, 1H, NH), 7.72-7.27 (m, 7H), 7.22-7.18 (m, 2H), 4.90 (d, J=6.0 Hz, 2H), 3.98 (s, 3H).

### Example 50: 3-[4-methoxy-2-(trifluoromethyl)phenyl]-N-[(1-oxidopyridin-1-ium-2-yl)methyl]benzamide

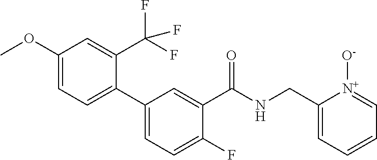 [SMILES: COc1ccc(-c2ccc(F)c(C(=O)NCc3cccc[n+]3[O-])c2)c(C(F)(F)F)c1]

The procedure given in Example 1 was followed using 4-methoxy-2-trifluoromethylphenyl boronic acid as a reactant, instead of 4-trifluoromethylphenyl boronic acid, to give 3-[4-methoxy-2-(trifluoromethyl)phenyl]-N-[(1-oxidopyridin-1-ium-2-yl)methyl]benzamide.

1H-NMR (CDCl₃, 500 MHz) δ=8.25 (d, J=6.0 Hz, 1H), 7.91-7.80 (m, 2H), 7.73 (s, 1H), 7.54 (dd, J=2.0, 7.5 Hz, 1H), 7.43 (d, J=5.5 Hz, 2H), 7.31-7.21 (m, 4H), 7.07 (dd, J=2.5, 8.5 Hz, 1H), 4.83 (d, J=6.0 Hz, 2H), 3.89 (s, 3H).

### Example 51: 5-(4-chlorophenyl)-2-fluoro-N-[(1-oxidopyridin-1-ium-2-yl)methyl]benzamide

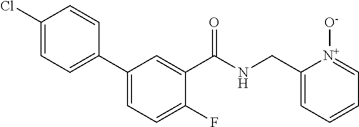 [SMILES: O=C(NCc1cccc[n+]1[O-])c1cc(-c2ccc(Cl)cc2)ccc1F]

The procedure given in Example 1 was followed using 4-chlorophenyl boronic acid and 5-bromo-2-fluoro-N-[(1-oxidopyridin-1-ium-2-yl)methyl]benzamide as a reactant, instead of 4-trifluoromethylphenyl boronic acid and 3-bromo-N-[(1-oxidopyridin-1-ium-2-yl)methyl]benzamide, respectively, to give 5-(4-chlorophenyl)-2-fluoro-N-[(1-oxidopyridin-1-ium-2-yl)methyl]benzamide.

1H-NMR (CDCl₃, 500 MHz) δ=8.25-8.19 (m, 2H), 8.18 (brs, 1H, NH), 7.63 (dq, J=2.5, 4.5 Hz, 1H), 7.55 (d, J=2.0 Hz, 1H), 7.53 (d, J=2.0 Hz, 2H), 7.49 (d, J=8.5 Hz, 2H), 7.41-7.29 (m, 2H), 7.21-7.17 (m, 1H), 4.88 (d, J=6.0 Hz, 2H).

### Example 52: 5-(3-chlorophenyl)-2-fluoro-N-[(1-oxidopyridin-1-ium-2-yl)methyl]benzamide

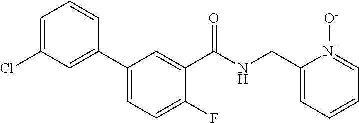 [SMILES: O=C(NCc1cccc[n+]1[O-])c1cc(-c2cccc(Cl)c2)ccc1F]

The procedure given in Example 51 was followed using 3-chlorophenyl boronic acid as a reactant, instead of 4-chlorophenyl boronic acid, to give 5-(3-chlorophenyl)-2-fluoro-N-[(1-oxidopyridin-1-ium-2-yl)methyl]benzamide.

1H-NMR (CDCl₃, 500 MHz) δ=8.28-8.24 (m, 3H), 7.65-7.18 (m, 9H), 4.88 (d, J=6.0 Hz, 2H).

### Example 53: 5-(4-chloro-2-methoxy-phenyl)-2-fluoro-N-[(1-oxidopyridin-1-ium-2-yl)methyl]benzamide

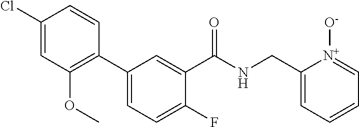 [SMILES: COc1cc(Cl)ccc1-c1ccc(F)c(C(=O)NCc2cccc[n+]2[O-])c1]

The procedure given in Example 51 was followed using 4-chloro-2-methoxyphenyl boronic acid as a reactant, instead of 4-chlorophenyl boronic acid, to give 5-(4-chloro-2-methoxy-phenyl)-2-fluoro-N-[(1-oxidopyridin-1-ium-2-yl)methyl]benzamide.

1H-NMR (CDCl₃, 500 MHz) δ=8.27 (d, J=6.0 Hz, 1H), 8.14-8.12 (m, 2H), 7.53-7.51 (m, 2H), 7.28-7.26 (m, 2H), 7.20 (d, J=8.0 Hz, 1H), 7.15 (t, J=3.0 Hz, 1H), 6.99 (dd, J=1.5, 5.0 Hz, 1H), 6.94 (d, J=2.0 Hz, 1H), 4.87 (d, J=6.0 Hz, 2H), 3.79 (s, 3H).

### Example 54: 5-(3,4-difluorophenyl)-2-fluoro-N-[(1-oxidopyridin-1-ium-2-yl)methyl]benzamide

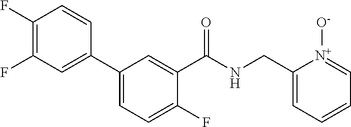 [SMILES: O=C(NCc1cccc[n+]1[O-])c1cc(-c2ccc(F)c(F)c2)ccc1F]

The procedure given in Example 51 was followed using 3,4-difluorophenyl boronic acid as a reactant, instead of 4-chlorophenyl boronic acid, to give 5-(3,4-difluorophenyl)-2-fluoro-N-[(1-oxidopyridin-1-ium-2-yl)methyl]benzamide.

1H-NMR (CDCl₃, 500 MHz) δ=8.30-8.19 (m, 3H), 7.61-7.18 (m, 7H), 4.87 (d, J=6.0 Hz, 2H).

### Example 55: 5-[4-chloro-2-(trifluoromethyl)phenyl]-2-fluoro-N-[(1-oxidopyridin-1-ium-2-yl)methyl]benzamide

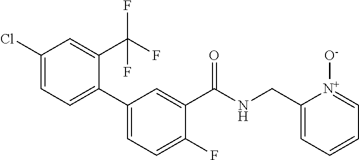 [SMILES: O=C(NCc1cccc[n+]1[O-])c1cc(-c2ccc(Cl)cc2C(F)(F)F)ccc1F]

The procedure given in Example 51 was followed using 4-chloro-2-trifluoromethylphenyl boronic acid as a reactant, instead of 4-chlorophenyl boronic acid, to give 5-[4-chloro-2-(trifluoromethyl)phenyl]-2-fluoro-N-[(1-oxidopyridin-1-ium-2-yl)methyl]benzamide.

1H-NMR (CDCl₃, 500 MHz) δ=8.29 (d, J=6.0 Hz, 1H), 8.27 (brs, 1H, NH), 7.99 (dd, J=2.5, 7.0 Hz, 1H), 7.73 (d, J=2.0 Hz, 1H), 7.54-7.23 (m, 7H), 7.17 (dd, J=8.5, 11.5 Hz, 1H), 4.86 (d, J=6.0 Hz, 2H).

### Example 56: 2-fluoro-5-[2-methoxy-4-(trifluoromethoxy)phenyl]-N-[(1-oxidopyridin-1-ium-2-yl)methyl]benzamide

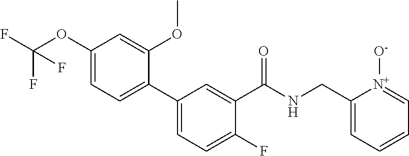 [SMILES: COc1cc(OC(F)(F)F)ccc1-c1ccc(F)c(C(=O)NCc2cccc[n+]2[O-])c1]

The procedure given in Example 51 was followed using 2-methoxy-4-trifluoromethoxyphenyl boronic acid as a reactant, instead of 4-chlorophenyl boronic acid, to give 2-fluoro-5-[2-methoxy-4-(trifluoromethoxy)phenyl]-N-[(1-oxidopyridin-1-ium-2-yl)methyl]benzamide.

1H-NMR (CDCl₃, 500 MHz) δ=8.26 (d, J=1.5 Hz, 1H), 8.13 (dd, J=2.5, 7.5 Hz, 2H), 7.58 (dt, J=2.5, 5.0 Hz, 1H), 7.51 (dd, J=2.5, 7.5 Hz, 1H), 7.29-7.25 (m, 3H), 7.15 (dd, J=9.0, 12.0 Hz, 1H), 6.87 (dd, J=1.0, 8.5 Hz, 1H), 6.80 (d, J=2.0 Hz, 1H), 4.86 (d, J=6.0 Hz, 2H), 3.80 (s, 3H).

### Example 57: 2-fluoro-N-[(1-oxidopyridin-1-ium-2-yl)methyl]-5-[4-(trifluoromethyl)phenyl]benzamide

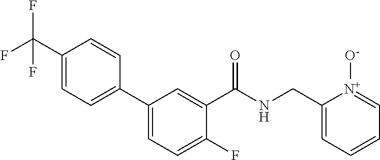 [SMILES: O=C(NCc1cccc[n+]1[O-])c1cc(-c2ccc(C(F)(F)F)cc2)ccc1F]

The procedure given in Example 51 was followed using 4-trifluoromethylphenyl boronic acid as a reactant, instead of 4-chlorophenyl boronic acid, to give 2-fluoro-N-[(1-oxidopyridin-1-ium-2-yl)methyl]-5-[4-(trifluoromethyl)phenyl]benzamide.

1H-NMR (CDCl₃, 500 MHz) δ=8.24-8.22 (m, 3H), 7.64-7.61 (m, 5H), 7.49 (dd, J=2.0, 7.5 Hz, 1H), 7.24-7.15 (m, 3H), 4.84 (d, J=6.0 Hz, 2H).

### Example 58: 2-fluoro-N-[(1-oxidopyridin-1-ium-2-yl)methyl]-5-[4-(trifluoromethoxy)phenyl]benzamide

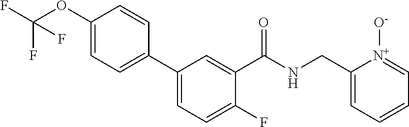 [SMILES: O=C(NCc1cccc[n+]1[O-])c1cc(-c2ccc(OC(F)(F)F)cc2)ccc1F]

The procedure given in Example 51 was followed using 4-trifluoromethoxyphenyl boronic acid as a reactant, instead of 4-chlorophenyl boronic acid, to give 2-fluoro-N-[(1-oxidopyridin-1-ium-2-yl)methyl]-5-[4-(trifluoromethoxy)phenyl]benzamide.

1H-NMR (CDCl₃, 500 MHz) δ=8.28-8.18 (m, 3H), 7.64-7.53 (m, 4H), 7.32-7.18 (m, 5H), 4.89 (d, J=5.0 Hz, 2H).

### Example 59: 2-fluoro-5-[3-methoxy-4-(trifluoromethyl)phenyl]-N-[(1-oxidopyridin-1-ium-2-yl)methyl]benzamide

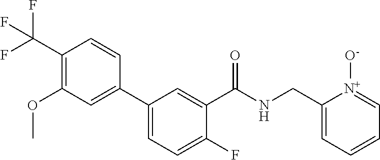 [SMILES: COc1cc(-c2ccc(F)c(C(=O)NCc3cccc[n+]3[O-])c2)ccc1C(F)(F)F]

The procedure given in Example 51 was followed using 3-methoxy-4-trifluoromethylphenyl boronic acid as a reactant, instead of 4-chlorophenyl boronic acid, to give 2-fluoro-5-[3-methoxy-4-(trifluoromethyl)phenyl]-N-[(1-oxidopyridin-1-ium-2-yl)methyl]benzamide.

1H-NMR (CDCl₃, 500 MHz) δ=8.29-8.21 (m, 3H), 7.68-7.53 (m, 3H), 7.32-7.16 (m, 4H), 7.14 (s, 1H), 4.89 (d, J=6.0 Hz, 2H), 3.96 (s, 3H).

### Example 60: 2-fluoro-5-[3-fluoro-4-(trifluoromethyl)phenyl]-N-[(1-oxidopyridin-1-ium-2-yl)methyl]benzamide

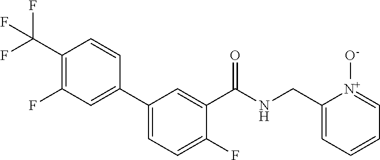 [SMILES: O=C(NCc1cccc[n+]1[O-])c1cc(-c2ccc(C(F)(F)F)c(F)c2)ccc1F]

The procedure given in Example 51 was followed using 3-fluoro-4-trifluoromethylphenyl boronic acid as a reactant, instead of 4-chlorophenyl boronic acid, to give 2-fluoro-5-[3-fluoro-4-(trifluoromethyl)phenyl]-N-[(1-oxidopyridin-1-ium-2-yl)methyl]benzamide.

1H-NMR (CDCl₃, 500 MHz) δ=8.29-8.23 (m, 3H), 7.67-7.38 (m, 5H), 7.31-7.21 (m, 3H), 4.86 (d, J=5.5 Hz, 2H).

### Example 61: 3-(4-chlorophenyl)-N-[(5-fluoro-1-oxido-pyridin-1-ium-2-yl)methyl]benzamide

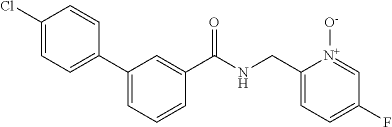 [SMILES: O=C(NCc1ccc(F)c[n+]1[O-])c1cccc(-c2ccc(Cl)cc2)c1]

The procedure given in Example 51 was followed using 3-(4-chlorophenyl)-N-[(5-fluoro-2-pyridyl)methyl]benzamide 3-bromo-5-fluoro-N-[(1-oxidopyridin-1-ium-2-yl)methyl]benzamide as a reactant, instead of 5-bromo-2-fluoro-N-[(1-oxidopyridin-1-ium-2-yl)methyl]benzamide, to give 3-(4-chlorophenyl)-N-[(5-fluoro-1-oxido-pyridin-1-ium-2-yl)methyl]benzamide.

1H-NMR (CDCl₃, 500 MHz) δ=8.19 (dd, J=2.5, 4.0 Hz, 1H), 7.99 (d, J=1.5 Hz, 1H), 7.75 (d, J=1.0 Hz, 1H), 7.73 (brs, 1H, NH), 7.69-7.41 (m, 7H), 7.13-7.09 (m, 1H), 4.81 (d, J=6.0 Hz, 2H).

### Example 62: 2-chloro-5-(4-chlorophenyl)-N-[(1-oxidopyridin-1-ium-2-yl)methyl]benzamide

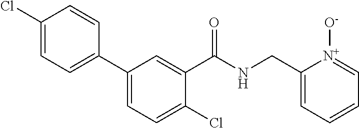 [SMILES: O=C(NCc1cccc[n+]1[O-])c1cc(-c2ccc(Cl)cc2)ccc1Cl]

The procedure given in Example 51 was followed using 5-bromo-2-chloro-N-[(1-oxidopyridin-1-ium-2-yl)methyl]benzamide as a reactant, instead of 5-bromo-2-fluoro-N-[(1-oxidopyridin-1-ium-2-yl)methyl]benzamide, to give 2-chloro-5-(4-chlorophenyl)-N-[(1-oxidopyridin-1-ium-2-yl)methyl]benzamide.

1H-NMR (DMSO-d₆, 500 MHz) δ=9.17 (t, J=7.5 Hz, 1H), 8.34-8.32 (m, 2H), 7.88 (d, J=3.0 Hz, 1H), 7.81-7.37 (m, 7H), 4.56 (d, J=7.5 Hz, 2H).

hNav1.7 Channel Assay

Whole cell patch clamp technique was used to investigate the inhibitory effects of the compound of Examples on human Nav1.7 channels, stably expressed in HEK293 cells. Human Nav1.7 recombinant cell line (CYL3011-precisION) was purchased from Eurofins.

Two-pulse protocol was used every 10 seconds to assess the state-dependent inhibition by Examples. From the holding potential of −90 mV, pre-pulse (200 ms at −120 mV) was followed by test pulse1 (TP1: is at 0 mV), then test pulse2 (TP2: 20 ms at 0 mV) was evoked after inter-pulse (20 ms at −100 mV).

Peak current amplitudes were measured for test pulses TP1 (resting phase inhibition) and TP2 (inactivated phase inhibition). The following solutions were used for screening assay: External (mM): 145 NaCl, 1 MgCl₂, 1.8 CaCl₂, 10 Hepes, 5 Glucose, pH 7.4, 300 mOsm; Internal (mM): 120 CsF, 10 NaCl, 10 EGTA, 10 HEPES, pH 7.2, 290 mOsm.

The inhibition on the inactivated state of hNav1.7 was calculated as: % inhibition (inactivation state)=(1−(I_{TP2}, TA/I_{TP2}, Control)×100%, where I_{TP2} (the inward peak Na+ currents), Control and I_{TP2}, TA were elicited by the TP2 in control (vehicle only) and in the presence of a test article (TA) indicating Examples, respectively.

According to one embodiment of the present invent, compounds of examples did not show significant inhibitions on the resting state of hNav1.7, indicating relatively lower adverse potentials of Examples.

TABLE 1

| Examples | Inhibition activity |
|---|---|
| Example 1 | +++ |
| Example 2 | +++ |
| Example 3 | +++ |
| Example 4 | ++ |
| Example 5 | ++ |
| Example 6 | +++ |
| Example 7 | ++ |
| Example 8 | ++ |
| Example 9 | +++ |
| Example 10 | ++ |
| Example 11 | +++ |
| Example 12 | ++ |
| Example 13 | ++ |
| Example 14 | ++ |
| Example 15 | ++ |
| Example 16 | ++ |
| Example 17 | ++ |
| Example 18 | ++ |
| Example 19 | ++ |
| Example 20 | ++ |
| Example 21 | ++ |
| Example 22 | ++ |
| Example 23 | ++ |
| Example 24 | + |
| Example 25 | +++ |
| Example 26 | + |
| Example 28 | +++ |
| Example 29 | +++ |
| Example 30 | ++ |
| Example 31 | ++ |
| Example 32 | +++ |
| Example 33 | + |
| Example 34 | + |
| Example 35 | +++ |
| Example 36 | ++ |
| Example 38 | ++ |
| Example 39 | +++ |
| Example 40 | +++ |
| Example 41 | + |
| Example 42 | ++ |
| Example 43 | +++ |
| Example 44 | +++ |
| Example 45 | + |
| Example 46 | ++ |
| Example 47 | + |
| Example 48 | ++ |
| Example 49 | +++ |
| Example 50 | ++ |
| Example 51 | +++ |
| Example 52 | +++ |
| Example 53 | +++ |
| Example 54 | +++ |
| Example 55 | ++ |
| Example 56 | +++ |
| Example 57 | +++ |
| Example 58 | +++ |
| Example 59 | +++ |
| Example 60 | +++ |
| Example 61 | +++ |
| Example 62 | +++ |

Inhibition activity means percent inhibition on the inactivated state of hNav1.7 at 10 μM.

$$+ : 10 - 40 \% , ++ : 40 - 70 \% , ++ + : 70 - 100 \%$$

As described herein, the compounds of the present disclosure were observed to have inhibiting affinity against voltage-dependent sodium channels. The results indicate that the compounds can be useful to treat pain including neuropathic pain by blocking the activities of sodium channels.

### Rat Spinal Nerve Ligation Model

Analgesic efficacy was examined through the Spinal Nerve Ligation (SNL) model, also known as the Chung model (Kim and Chung, 1992). SNL model is one of the most frequently used animal models for the assessment of neuropathic pain behavior.

Briefly, the lumbar 5 spinal nerve is ligated with silk suture distal to the dorsal root ganglion. After two weeks of recovery, mechanical allodynia in the affected paw of animals was evaluated using von Frey filaments. Using the Dixons Up-Down method (Chaplan et al, 1994), a pre-drug baseline was taken with allodynia defined as a paw withdrawal threshold (PWT)<4 g. Test compounds (at 30 mg/kg) or vehicle were then orally administered, and PWT was determined two hours after the treatment. Statistical mean values of PWT for vehicle control and drug treatment groups were calculated, and the percent of maximum possible effect (% MPE) value was determined according to the following formula; % MPE=[(post-drug PWT−pre-drug PWT)/(cutoff PWT−pre-drug PWT)]×100.

TABLE 2

| Example | in vivo efficacy |
|---|---|
| Example 2 | +++ |
| Example 3 | ++ |
| Example 14 | ++ |
| Example 19 | ++ |
| Example 22 | + |
| Example 51 | +++ |

in vivo efficacy means % MPE at 30 mg/kg, p.o.
+: 10-40%,
++: 40-70%,
+++: 70-100%

As described above, the compounds of the present disclosure were observed to have analgesic efficacy which is useful for the treatment of pain including neuropathic pain.

1. A compound of the following Formula (I), or an optical isomer, a stereoisomer or an isotopic variant thereof, or a pharmaceutically acceptable salt thereof: 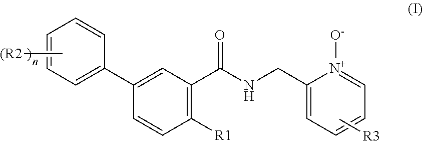
wherein
R1 is hydrogen, halogen, alkyl, alkoxy or cycloalkyl;
R2 is hydrogen, halogen, alkyl, alkoxy, cycloalkyl or 4- to 10-membered heterocycloalkyl which includes one or more heteroatoms selected from N, O and S;
R3 is hydrogen, halogen, alkyl, alkoxy or cycloalkyl; and
n is an integer of 0 to 4,
wherein the alkyl, alkoxy and cycloalkyl are optionally substituted with one or more substituents selected from halogen and hydroxy.

2. The compound, or optical isomer, stereoisomer or isotopic variant thereof, or a pharmaceutically acceptable salt thereof according to claim 1, wherein
R1 is hydrogen, halogen, C₁-C₆ alkyl, C₁-C₆ alkoxy or C₃-C₁₀ cycloalkyl;
R2 is hydrogen, halogen, C₁-C₆ alkyl, C₁-C₆ alkoxy, C₃-C₁₀ cycloalkyl or 4- to 10-membered heterocycloalkyl which includes 1 to 4 heteroatoms selected from N, O and S;
R3 is hydrogen, halogen, C₁-C₆ alkyl, C₁-C₆ alkoxy or C₃-C₁₀ cycloalkyl; and
n is an integer of 0 to 4,
wherein the alkyl, alkoxy and cycloalkyl are optionally substituted with 1 to 4 substituents selected from halogen and hydroxy.

3. The compound, or optical isomer, stereoisomer or isotopic variant thereof, or a pharmaceutically acceptable salt thereof according to claim 1, wherein
R1 is hydrogen, halogen, C₁-C₄ alkyl, C₁-C₄ alkoxy or C₃-C₆ cycloalkyl;
R2 is hydrogen, halogen, C₁-C₄ alkyl, C₁-C₄ alkoxy, C₃-C₆ cycloalkyl or 4- to 10-membered heterocycloalkyl which includes 1 to 4 heteroatoms selected from N, O and S;
R3 is hydrogen, halogen, C₁-C₄ alkyl, C₁-C₄ alkoxy or C₃-C₆ cycloalkyl; and
n is an integer of 0 to 4,
wherein the alkyl, alkoxy and cycloalkyl are optionally substituted with 1 to 4 substituents selected from halogen and hydroxy.

4. The compound, or optical isomer, stereoisomer or isotopic variant thereof, or a pharmaceutically acceptable salt thereof according to claim 1, wherein
R1 is hydrogen or halogen.

5. The compound, or optical isomer, stereoisomer or isotopic variant thereof, or a pharmaceutically acceptable salt thereof according to claim 1, wherein
R2 is hydrogen, halogen, C₁-C₆ alkyl, C₁-C₆ alkoxy or C₃-C₁₀ cycloalkyl;
wherein the alkyl, alkoxy and cycloalkyl are optionally substituted with 1 to 4 substituents selected from halogen and hydroxy.

6. The compound, or optical isomer, stereoisomer or isotopic variant thereof, or a pharmaceutically acceptable salt thereof according to claim 1, wherein
R3 is hydrogen or halogen.

7. The compound, or optical isomer, stereoisomer or isotopic variant thereof, or a pharmaceutically acceptable salt thereof according to claim 1, wherein the compound of Formula (I) is a compound of Formula (II): 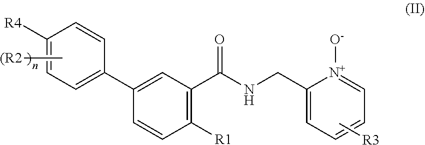
wherein
R1 is hydrogen, halogen, alkyl, alkoxy or cycloalkyl;
R2 is hydrogen, halogen, alkyl, alkoxy, cycloalkyl or 4- to 10-membered heterocycloalkyl which includes one or more heteroatoms selected from N, O and S;
R3 is hydrogen, halogen, alkyl, alkoxy or cycloalkyl;
R4 is hydrogen, halogen, alkyl, alkoxy, cycloalkyl or 4- to 10-membered heterocycloalkyl which includes one or more heteroatoms selected from N, O and S; and
n is an integer of 0 to 3,
wherein the alkyl, alkoxy and cycloalkyl are optionally substituted with one or more substituents selected from halogen and hydroxy.

8. The compound, or optical isomer, stereoisomer or isotopic variant thereof, or a pharmaceutically acceptable salt thereof according to claim 7, wherein
R1 is hydrogen, halogen, C₁-C₆ alkyl, C₁-C₆ alkoxy or C₃-C₁₀ cycloalkyl;
R2 is hydrogen, halogen, C₁-C₆ alkyl, C₁-C₆ alkoxy, C₃-C₁₀ cycloalkyl or 4- to 10-membered heterocycloalkyl which includes 1 to 4 heteroatoms selected from N, O and S;
R3 is hydrogen, halogen, C₁-C₆ alkyl, C₁-C₆ alkoxy or C₃-C₁₀ cycloalkyl;
R4 is hydrogen, halogen, C₁-C₆ alkyl, C₁-C₆ alkoxy, C₃-C₁₀ cycloalkyl or 4- to 10-membered heterocycloalkyl which includes 1 to 4 heteroatoms selected from N, O and S; and
n is an integer of 0 to 3,
wherein the alkyl, alkoxy and cycloalkyl are optionally substituted with 1 to 4 substituents selected from halogen and hydroxy.

9. The compound, or optical isomer, stereoisomer or isotopic variant thereof, or a pharmaceutically acceptable salt thereof according to claim 7, wherein
R1 is hydrogen, halogen, C₁-C₄ alkyl, C₁-C₄ alkoxy or C₃-C₆ cycloalkyl;
R2 is hydrogen, halogen, C₁-C₄ alkyl, C₁-C₄ alkoxy, C₃-C₆ cycloalkyl or 4- to 10-membered heterocycloalkyl which includes 1 to 4 heteroatoms selected from N, O and S;
R3 is hydrogen, halogen, C₁-C₄ alkyl, C₁-C₄ alkoxy or C₃-C₆ cycloalkyl;
R4 is hydrogen, halogen, C₁-C₄ alkyl, C₁-C₄ alkoxy, C₃-C₆ cycloalkyl or 4- to 10-membered heterocycloalkyl which includes 1 to 4 heteroatoms selected from N, O and S; and
n is an integer of 0 to 3,
wherein the alkyl, alkoxy and cycloalkyl are optionally substituted with 1 to 4 substituents selected from halogen and hydroxy.

10. The compound, or optical isomer, stereoisomer or isotopic variant thereof, or a pharmaceutically acceptable salt thereof according to claim 7, wherein
R1 is hydrogen or halogen.

11. The compound, or optical isomer, stereoisomer or isotopic variant thereof, or a pharmaceutically acceptable salt thereof according to claim 7, wherein
R2 is hydrogen, halogen, C₁-C₆ alkyl or C₁-C₆ alkoxy;
wherein the alkyl and alkoxy are optionally substituted with 1 to 4 substituents selected from halogen and hydroxy.

12. The compound, or optical isomer, stereoisomer or isotopic variant thereof, or a pharmaceutically acceptable salt thereof according to claim 7, wherein
R3 is hydrogen or halogen.

13. The compound, or optical isomer, stereoisomer or isotopic variant thereof, or a pharmaceutically acceptable salt thereof according to claim 7, wherein
R4 is hydrogen, halogen, C₁-C₆ alkyl, C₁-C₆ alkoxy or C₃-C₁₀ cycloalkyl;
wherein the alkyl, alkoxy and cycloalkyl are optionally substituted with 1 to 4 substituents selected from halogen and hydroxy.

14. The compound, or optical isomer, stereoisomer or isotopic variant thereof, or a pharmaceutically acceptable salt thereof according to claim 1, which is selected from the group consisting of:
N-[(1-oxidopyridin-1-ium-2-yl)methyl]-3-[4-(trifluoromethyl)phenyl]benzamide;
3-(4-chlorophenyl)-N-[(1-oxidopyridin-1-ium-2-yl)methyl]benzamide;
N-[(1-oxidopyridin-1-ium-2-yl)methyl]-3-[4-(trifluoromethoxy)phenyl]benzamide;
3-(3-chlorophenyl)-N-[(1-oxidopyridin-1-ium-2-yl)methyl]benzamide;
N-[(1-oxidopyridin-1-ium-2-yl)methyl]-3-[3-(trifluoromethyl)phenyl]benzamide;
3-(3,4-dichlorophenyl)-N-[(1-oxidopyridin-1-ium-2-yl)methyl]benzamide;
3-(3,5-dichlorophenyl)-N-[(1-oxidopyridin-1-ium-2-yl)methyl]benzamide;
N-[(1-oxidopyridin-1-ium-2-yl)methyl]-3-[3-(trifluoromethoxy)phenyl]benzamide;
3-(2,4-dichlorophenyl)-N-[(1-oxidopyridin-1-ium-2-yl)methyl]benzamide;
3-(2,5-dichlorophenyl)-N-[(1-oxidopyridin-1-ium-2-yl)methyl]benzamide;
3-[2,4-bis(trifluoromethyl)phenyl]-N-[(1-oxidopyridin-1-ium-2-yl)methyl]benzamide;
N-[(1-oxidopyridin-1-ium-2-yl)methyl]-3-phenyl-benzamide;
3-(4-tert-butylphenyl)-N-[(1-oxidopyridin-1-ium-2-yl)methyl]benzamide;
N-[(1-oxidopyridin-1-ium-2-yl)methyl]-3-(p-tolyl)benzamide;
3-(4-chloro-2-methyl-phenyl)-N-[(1-oxidopyridin-1-ium-2-yl)methyl]benzamide;
3-[2-fluoro-4-(trifluoromethyl)phenyl]-N-[(1-oxidopyridin-1-ium-2-yl)methyl]benzamide;
3-(3-chloro-4-fluoro-phenyl)-N-[(1-oxidopyridin-1-ium-2-yl)methyl]benzamide;
3-(4-fluorophenyl)-N-[(1-oxidopyridin-1-ium-2-yl)methyl]benzamide;
3-(3,4-difluorophenyl)-N-[(1-oxidopyridin-1-ium-2-yl)methyl]benzamide;
3-(2-chlorophenyl)-N-[(1-oxidopyridin-1-ium-2-yl)methyl]benzamide;
N-[(1-oxidopyridin-1-ium-2-yl)methyl]-3-[2-(trifluoromethyl)phenyl]benzamide;
3-(4-fluoro-3-methyl-phenyl)-N-[(1-oxidopyridin-1-ium-2-yl)methyl]benzamide;
3-(m-tolyl)-N-[(1-oxidopyridin-1-ium-2-yl)methyl]benzamide;
3-(4-isopropylphenyl)-N-[(1-oxidopyridin-1-ium-2-yl)methyl]benzamide;
3-(2-fluorophenyl)-N-[(1-oxidopyridin-1-ium-2-yl)methyl]benzamide;
3-(4-chloro-2-methoxy-phenyl)-N-[(1-oxidopyridin-1-ium-2-yl)methyl]benzamide;
3-[4-chloro-2-(trifluoromethyl)phenyl]-N-[(1-oxidopyridin-1-ium-2-yl)methyl]benzamide;
3-(4-chloro-3-methyl-phenyl)-N-[(1-oxidopyridin-1-ium-2-yl)methyl]benzamide;
3-(3-chloro-5-fluoro-phenyl)-N-[(1-oxidopyridin-1-ium-2-yl)methyl]benzamide;
3-(4-chloro-3-fluoro-phenyl)-N-[(1-oxidopyridin-1-ium-2-yl)methyl]benzamide;
3-(2,4-difluorophenyl)-N-[(1-oxidopyridin-1-ium-2-yl)methyl]benzamide;
3-(3,5-difluorophenyl)-N-[(1-oxidopyridin-1-ium-2-yl)methyl]benzamide;
N-[(1-oxidopyridin-1-ium-2-yl)methyl]-3-(3,4,5-trifluorophenyl)benzamide;
3-(2,3-difluorophenyl)-N-[(1-oxidopyridin-1-ium-2-yl)methyl]benzamide;
3-(3-isopropylphenyl)-N-[(1-oxidopyridin-1-ium-2-yl)methyl]benzamide;
3-(4-cyclopropylphenyl)-N-[(1-oxidopyridin-1-ium-2-yl)methyl]benzamide;
3-[4-(difluoromethoxy)phenyl]-N-[(1-oxidopyridin-1-ium-2-yl)methyl]benzamide;
3-[2-methyl-4-(trifluoromethyl)phenyl]-N-[(1-oxidopyridin-1-ium-2-yl)methyl]benzamide;
3-[4-fluoro-3-(trifluoromethyl)phenyl]-N-[(1-oxidopyridin-1-ium-2-yl)methyl]benzamide;
3-(2-methoxyphenyl)-N-[(1-oxidopyridin-1-ium-2-yl)methyl]benzamide;
3-[3-fluoro-5-(trifluoromethyl)phenyl]-N-[(1-oxidopyridin-1-ium-2-yl)methyl]benzamide;
3-[3-fluoro-4-(trifluoromethyl)phenyl]-N-[(1-oxidopyridin-1-ium-2-yl)methyl]benzamide;
3-[3-methyl-4-(trifluoromethyl)phenyl]-N-[(1-oxidopyridin-1-ium-2-yl)methyl]benzamide;
3-[4-(1-hydroxy-1-methyl-ethyl)phenyl]-N-[(1-oxidopyridin-1-ium-2-yl)methyl]benzamide;
3-[3-methoxy-5-(trifluoromethyl)phenyl]-N-[(1-oxidopyridin-1-ium-2-yl)methyl]benzamide;
3-[2-methoxy-4-(trifluoromethoxy)phenyl]-N-[(1-oxidopyridin-1-ium-2-yl)methyl]benzamide;
3-(4,5-difluoro-2-methoxy-phenyl)-N-[(1-oxidopyridin-1-ium-2-yl)methyl]benzamide;
3-(4-chloro-3-methoxy-phenyl)-N-[(1-oxidopyridin-1-ium-2-yl)methyl]benzamide;
3-[3-methoxy-4-(trifluoromethyl)phenyl]-N-[(1-oxidopyridin-1-ium-2-yl)methyl]benzamide;
3-[4-methoxy-2-(trifluoromethyl)phenyl]-N-[(1-oxidopyridin-1-ium-2-yl)methyl]benzamide;
5-(4-chlorophenyl)-2-fluoro-N-[(1-oxidopyridin-1-ium-2-yl)methyl]benzamide;
5-(3-chlorophenyl)-2-fluoro-N-[(1-oxidopyridin-1-ium-2-yl)methyl]benzamide;
5-(4-chloro-2-methoxy-phenyl)-2-fluoro-N-[(1-oxidopyridin-1-ium-2-yl)methyl]benzamide;
5-(3,4-difluorophenyl)-2-fluoro-N-[(1-oxidopyridin-1-ium-2-yl)methyl]benzamide;
5-[4-chloro-2-(trifluoromethyl)phenyl]-2-fluoro-N-[(1-oxidopyridin-1-ium-2-yl)methyl]benzamide;
2-fluoro-5-[2-methoxy-4-(trifluoromethoxy)phenyl]-N-[(1-oxidopyridin-1-ium-2-yl)methyl]benzamide;
2-fluoro-N-[(1-oxidopyridin-1-ium-2-yl)methyl]-5-[4-(trifluoromethyl)phenyl]benzamide;
2-fluoro-N-[(1-oxidopyridin-1-ium-2-yl)methyl]-5-[4-(trifluoromethoxy)phenyl]benzamide;
2-fluoro-5-[3-methoxy-4-(trifluoromethyl)phenyl]-N-[(1-oxidopyridin-1-ium-2-yl)methyl]benzamide;
2-fluoro-5-[3-fluoro-4-(trifluoromethyl)phenyl]-N-[(1-oxidopyridin-1-ium-2-yl)methyl]benzamide;
3-(4-chlorophenyl)-N-[(5-fluoro-1-oxido-pyridin-1-ium-2-yl)methyl]benzamide; and
2-chloro-5-(4-chlorophenyl)-N-[(1-oxidopyridin-1-ium-2-yl)methyl]benzamide.

15. A pharmaceutical composition comprising a therapeutically effective amount of the compound, or optical isomer, stereoisomer or isotopic variant thereof, or a pharmaceutically acceptable salt thereof as defined in claim 1, together with a pharmaceutically acceptable carrier.

16. The pharmaceutical composition according to claim 15, which is for treating or preventing a disease or condition mediated by sodium channels.

17. The pharmaceutical composition according to claim 16, wherein the sodium channel is one or more selected from the group of consisting of NaV1.1, NaV1.2, NaV1.3, NaV 1.4, NaV1.5, NaV 1.6, NaV 1.7, NaV 1.8, and NaV1.9.

18. The pharmaceutical composition according to claim 16, wherein the disease or condition is pain.

19. The pharmaceutical composition according to claim 18, wherein the pain is selected from the group consisting of acute pain, chronic pain, neuropathic pain, inflammatory pain, visceral pain, nociceptive pain including post-surgical pain, and mixed pain types involving the viscera, gastrointestinal tract, cranial structures, musculoskeletal system, spine, urogenital system, cardiovascular system and CNS (central nervous system), including cancer pain, back pain, orofacial pain and chemo-induced pain.

20. The pharmaceutical composition according to claim 16, wherein the disease or condition is selected from the group consisting of neurological disorders, neurodegenerative disorders, inflammatory disorders, gastrointestinal (GI) tract disorders, disorders of the genito-urinary tract, psychiatric disorders, cardiovascular disorders and neuromuscular disorders.

21. The pharmaceutical composition according to claim 15, which is an oral formulation.

22. The pharmaceutical composition according to claim 15, further comprising an active ingredient which is effective for treating or preventing pain including neuropathic pain.

23. The pharmaceutical composition of claim 22, wherein the active ingredient is selected from the group consisting of opioid receptor agonists or antagonist, sodium channel blocker, calcium channel blocker, potassium channel blocker, pregabalin, gabapentin, antidepressants, lithium and valproate.

24. A method of treating or preventing a disease or condition mediated by sodium channels, comprising:
administering a therapeutically effective amount of the compound, or optical isomer, stereoisomer or isotopic variant thereof, or a pharmaceutically acceptable salt thereof as defined in claim 1 to a mammal in need thereof.

25. Use of the compound, or optical isomer, stereoisomer or isotopic variant thereof, or a pharmaceutically acceptable salt thereof as defined in claim 1 in the manufacture of a medicament for treating or preventing a disease or condition mediated by sodium channels.
